(19) United States
(12) Patent Application Publication
(10) Pub. No.: US 2023/0240512 A1
(43) Pub. Date: Aug. 3, 2023

(54) ENDOSCOPE SYSTEM, MANIPULATION ASSISTANCE METHOD, AND MANIPULATION ASSISTANCE PROGRAM

(71) Applicant: OLYMPUS CORPORATION, Tokyo (JP)

(72) Inventors: Takeshi ARAI, Tokyo (JP); Masahiro FUJII, Tokyo (JP); Takeo UCHIDA, Tokyo (JP); Masayuki KOBAYASHI, Tokyo (JP); Noriaki YAMANAKA, Tokyo (JP); Hisatsugu TAJIMA, Tokyo (JP)

(73) Assignee: OLYMPUS CORPORATION, Tokyo (JP)

(21) Appl. No.: 18/118,342

(22) Filed: Mar. 7, 2023

Related U.S. Application Data
(63) Continuation of application No. PCT/JP2022/004206, filed on Feb. 3, 2022.
(60) Provisional application No. 63/145,580, filed on Feb. 4, 2021.

(51) Int. Cl. A61B 1/00; A61B 17/29; A61B 34/20

(52) U.S. Cl. CPC A61B 1/000095; A61B 17/29; A61B 1/00045; A61B 34/20; A61B 1/000094; A61B 2034/2065

(57) ABSTRACT

Provided is an endoscope system including an endoscope that images living tissue to be treated by at least one instrument; and a control device that includes a processor configured to derive at least one of the following information: grasp assistance information on a grasping operation to grasp the living tissue by the at least one instrument, and traction assistance information on a traction operation for traction of the living tissue by the at least one instrument, based on an endoscopic image acquired by the endoscope.

### CROSS-REFERENCE TO RELATED APPLICATIONS

The present application claims priority under the provisional U.S. patent application No. 63/145,580 filed on Feb. 4, 2021, which is incorporated herein by reference. This is a continuation of International Application PCT/JP2022/004206 which is hereby incorporated by reference herein in its entirety.

### TECHNICAL FIELD

The present invention relates to an endoscope system, a manipulation assistance method, and a manipulation assistance program.

### BACKGROUND ART

There have been known technologies to assist surgeons in performing manipulation operations by displaying treatment-related information on an image of a living tissue to be treated (see, for example, PTLs 1 and 2).

The technology described in PTL 1 aims at improvement in recognition of living tissue by recognizing regions of living tissue in real time using image information acquired by an endoscope and presenting the recognized regions on the endoscopic image. The technology described in PTL 2 controls surgical instruments according to the surgical scene based on machine-learned data, for example, by displaying a staple-prohibited zone on an organ image during use of a stapler to prevent the staples from being shot into the prohibited zone.

### CITATION LIST

### Patent Literature

{PTL 1} U.S. patent Ser. No. 10/307,209

### {PTL 2} Japanese Patent Laid-Open No. 2021-13722

### SUMMARY OF INVENTION

A first aspect of the present invention is an endoscope system including: an endoscope that images living tissue to be treated by at least one instrument; and a control device that includes a processor configured to derive at least one of the following information: grasp assistance information on a grasping operation to grasp the living tissue by the at least one instrument, and traction assistance information on a traction operation for traction of the living tissue by the at least one instrument, based on an endoscopic image acquired by the endoscope.

A second aspect of the present invention is a manipulation assistance method including deriving, based on a living tissue image of living tissue to be treated by an at least one instrument, at least one of the following information: grasp assistance information on a grasping operation to grasp the living tissue by the at least one instrument, and traction assistance information on a traction operation for traction of the living tissue by the at least one instrument.

A third aspect of the present invention is a non-transitory computer-readable medium having a manipulation assistance program stored therein, the program causing a computer to execute functions of: acquiring an image of living tissue to be treated by at least one instrument; and deriving, based on the acquired living tissue image, at least one of the following information: grasp assistance information on a grasping operation to grasp the living tissue by the at least one instrument, and traction assistance information on a traction operation for traction of the living tissue by the at least one instrument.

### BRIEF DESCRIPTION OF DRAWINGS

FIG. 1 is a schematic configuration diagram of an endoscope system according to the first embodiment of the present invention.

FIG. 2 is a schematic configuration diagram of a control device.

FIG. 3 is a diagram for explaining the first teacher data and evaluation region.

FIG. 4 is a diagram for explaining the second teacher data and traction assistance information.

FIG. 5 is a flowchart for explaining the manipulation assistance method according to the first embodiment.

FIG. 6 is a diagram for explaining the teacher data and traction assistance information according to the first modification of the first embodiment.

FIG. 7 is a diagram for explaining other teacher data and other traction assistance information according to the first modification.

FIG. 8 is a diagram for explaining the evaluation performed by an evaluation unit according to the third modification of the first embodiment.

FIG. 9 is a diagram for explaining the feature values of an endoscopic image according to the fourth modification of the first embodiment.

FIG. 10 is a diagram for explaining traction assistance information according to the fifth modification of the first embodiment.

FIG. 11 is a diagram for explaining the teacher data and traction assistance information according to the sixth modification of the first embodiment.

FIG. 12 is a schematic configuration diagram of an endoscope system according to the seventh modification of the first embodiment.

FIG. 13 is a diagram for explaining traction assistance information according to the seventh modification.

FIG. 14 is a diagram for explaining other traction assistance information according to the seventh modification.

FIG. 15 is a flowchart for explaining a manipulation assistance method according to the eighth modification of the first embodiment.

FIG. 16 is a diagram for explaining an endoscope system according to the second embodiment of the present invention.

FIG. 17 is a flowchart for explaining a manipulation assistance method according to the second embodiment.

FIG. 18 is a diagram for explaining the teacher data and grasp assistance information of the first modification of the second embodiment.

FIG. 19 is a diagram for explaining the teacher data and grasp assistance information according to the second modification of the second embodiment.

FIG. 20 is a flowchart for explaining a manipulation assistance method according to the third and fourth modifications of the second embodiment.

FIG. 21 is a diagram for explaining grasp assistance information according to the third modification of the second embodiment.

FIG. 22 is a diagram for explaining grasp assistance information according to the fourth modification of the second embodiment.

FIG. 23 is a diagram for explaining teacher data and grasp assistance information according to the fifth modification of the second embodiment.

FIG. 24 is a diagram for explaining the manipulation assistance method using an endoscope system according to the sixth modification of the second embodiment.

FIG. 25 is a flowchart for explaining a manipulation assistance method according to the sixth modification.

FIG. 26 is a diagram for explaining grasp assistance information according to the sixth modification.

FIG. 27 is a flowchart for explaining a manipulation assistance method according to the seventh modification of the second embodiment.

FIG. 28 is a diagram for explaining teacher data and grasp assistance information according to the seventh modification.

FIG. 29 is a diagram for explaining grasp assistance information according to the eighth modification of the second embodiment.

FIG. 30 is a diagram for explaining a manipulation assistance method according to the third embodiment of the present invention.

FIG. 31 is a schematic configuration diagram of an endoscope system according to the third embodiment.

FIG. 32 is a flowchart for explaining a manipulation assistance method according to the third embodiment.

FIG. 33 is a diagram for explaining a manipulation assistance method according to the first modification of the third embodiment.

FIG. 34 is a diagram for explaining a manipulation assistance method according to the second modification of the third embodiment.

FIG. 35 is a diagram for explaining a manipulation assistance method according to the third modification of the third embodiment.

FIG. 36 is a diagram for explaining a manipulation assistance method according to the fourth modification of the third embodiment.

FIG. 37 is a flowchart for explaining a manipulation assistance method according to the fourth embodiment.

FIG. 38 is a diagram for explaining a grasping scene recognition step.

FIG. 39 is a diagram for explaining a tissue relaxation navigation step.

FIG. 40 is a diagram for explaining a grasp recognition step.

FIG. 41 is a diagram for explaining how the grasp point moves with the relaxation of the living tissue.

FIG. 42 is a flowchart for explaining a manipulation assistance method according to the first modification of the fourth embodiment.

FIG. 43 is a flowchart for explaining a manipulation assistance method according to the second modification of the fourth embodiment.

FIG. 44 is a diagram for explaining the deformation of tissue due to traction.

FIG. 45 is a diagram for explaining the relationship between traction force and changes in living tissue.

FIG. 46 is a flowchart for explaining a manipulation assistance method according to the third modification of the fourth embodiment.

FIG. 47 is a diagram for explaining the direction of relaxation of living tissue.

FIG. 48 is a diagram showing an example of a sensor mounted on a forceps according to the fourth modification of the fourth embodiment.

FIG. 49 is a flowchart for explaining a manipulation assistance method according to the fourth modification.

FIG. 50 is a schematic configuration diagram of an endoscope system according to the fifth embodiment of the present invention.

FIG. 51 is a diagram of an endoscope system according to the fifth embodiment.

FIG. 52 is a flowchart for explaining a manipulation assistance method according to the fifth embodiment.

FIG. 53 is a diagram for explaining an endoscope system according to the first modification of the fifth embodiment.

FIG. 54 is a schematic configuration diagram of the endoscope system according to the first modification.

FIG. 55 is a flowchart for explaining a manipulation assistance method according to the first modification.

FIG. 56 is a diagram for explaining an endoscope system according to the second modification of the fifth embodiment.

FIG. 57 is a schematic configuration diagram of the endoscope system according to the second modification.

FIG. 58 is a flowchart for explaining a manipulation assistance method according to the second modification.

FIG. 59 is a diagram for explaining an endoscope system according to the third modification of the fifth embodiment.

FIG. 60 is a flowchart for explaining a manipulation assistance method according to the third modification.

FIG. 61 is a diagram for explaining the traction direction component and the amount of movement of surrounding tissue when living tissue is pulled with a single instrument.

FIG. 62 is a diagram for explaining changes in the distribution of the amount of movement of living tissue before and during traction.

FIG. 63 is a diagram for explaining changes in the distance between the centers of gravity of the distribution of the amount of movement of the living tissue and the instrument before and during traction.

FIG. 64 is a diagram for explaining the center of the image and the direction vector and flow vector of each pixel when living tissue is pulled with a plurality of instruments.

FIG. 65 is a diagram for explaining the phenomenon that a stronger traction force increases the exposure of small blood vessels in the superficial layer of the surrounding tissue.

FIG. 66 is a diagram for explaining the phenomenon that applying tension to living tissue increases bright spots on an endoscopic image.

FIG. 67 is a diagram showing an example of an endoscopic image and a power spectrum image for explaining the phenomenon that a stronger traction force results in more high-frequency components in the image.

FIG. 68 is a diagram for explaining radial distribution p(γ).

FIG. 69 is a diagram for explaining angular directional distribution q(θ).

FIG. 70 is a diagram for explaining the phenomenon that the surface shape of the living tissue changes before and after traction.

FIG. 71 is a diagram for explaining the phenomenon that the surface shape of the living tissue changes before and after traction.

FIG. 72 is a diagram for explaining the phenomenon that the stronger the traction force, the smaller the curvature of the edge of the incision region of the living tissue.

FIG. 73 is a diagram for explaining the phenomenon that the area, contour, and ridge shape of the stretch region change with the strength of the traction force.

FIG. 74 is a diagram for explaining the phenomenon that the stronger the traction force, the flatter the surface texture of the living tissue.

FIG. 75 is a diagram for explaining the deviation between the surface of the surrounding tissue and the approximate plane.

### DESCRIPTION OF EMBODIMENTS

### First Embodiment

An endoscope system, manipulation assistance method, and manipulation assistance program according to the first embodiment of the present invention will be described below with reference to the accompanying drawings.

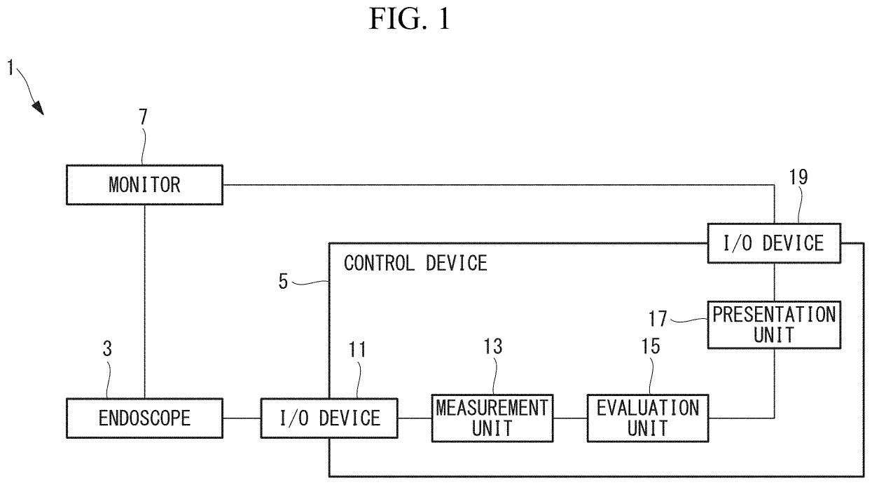

As shown in FIG. 1, the endoscope system 1 according to this embodiment includes an endoscope 3 for imaging tissues in a living body, a control device 5 that derives various information based on endoscopic images acquired with the endoscope 3, and a monitor (display device) 7 that displays endoscopic images and various information derived by the control device 5.

The control device 5 includes a first I/O device 11 for capturing endoscopic images acquired by the endoscope 3, a measurement unit 13 for measuring the feature values of the captured endoscopic images, an evaluation unit 15 for evaluating traction operation based on the measurement results given by the measurement unit 13, a presentation unit 17 for adding traction assistance information to the endoscopic images based on the evaluation results given by the evaluation unit 15, a second I/O device 19 for outputting the endoscopic images accompanied by the traction assistance information that has been added by the presentation unit 17, to the monitor 7.

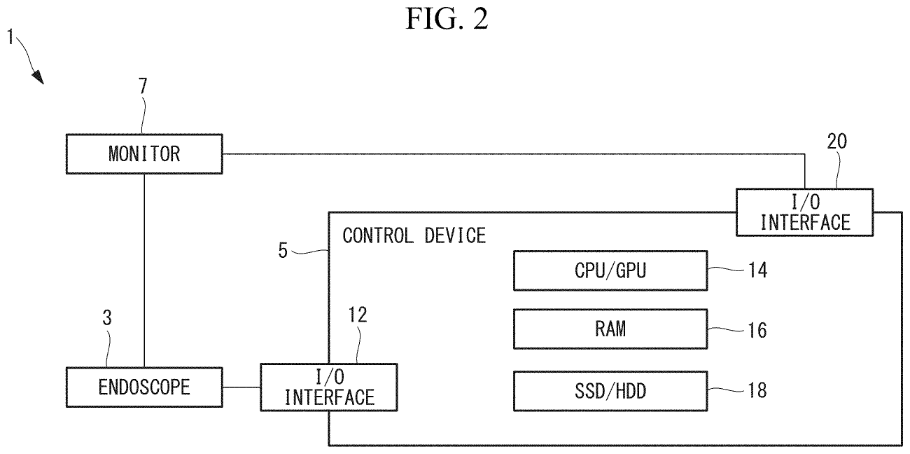

The control device 5 is implemented, for example, using a dedicated or general-purpose computer. As shown in FIG. 2, the control device 5 includes a first I/O interface 12 corresponding to the first I/O device 11, a processor 14, such as a central processing unit (CPU) or graphics processing unit (GPU), that constitutes the measurement unit 13, the evaluation unit 15, and the presentation unit 17, a main memory 16, such as a random access memory (RAM), that is used as a work area of the processor 14, an auxiliary memory 18, and a second I/O interface 20 corresponding to the second I/O device 19.

The auxiliary memory 18 is a computer-readable, non-transient storage medium, such as a solid state drive (SSD) or hard disk drive (HDD). The auxiliary memory 18 stores the manipulation assistance program whose processing is run by the processor 14 and various programs adjusted by machine learning. The main memory 16 and the auxiliary memory 18 may be connected to the control device 5 via a network.

The manipulation assistance program causes the control device 5 to perform an acquisition step of acquiring an image of living tissue being pulled by a jaw (instrument) 9 or the like; a derivation step of deriving traction assistance information related to the living tissue traction operation by the jaws 9 based on the acquired living tissue image; and a display step of displaying the derived traction assistance information in association with the living tissue image. As for the manipulation assistance program, each step of the manipulation assistance method, which will be described below, is executed by the control device 5.

When the processor 14 executes processing according to the manipulation assistance program, the functions of the measurement unit 13, evaluation unit 15, and presentation unit 17 are implemented. The computer constituting the control device 5 is connected to the endoscope 3, monitor 7, and input devices such as a mouse and keyboard (not shown in the drawing). The surgeon can use the input devices to input instructions necessary for image processing to the control device 5.

The measurement unit 13 measures feature values related to the grasp of the living tissue in the captured endoscopic image. The feature values related to the grasp of the living tissue are, for example, the tissue grasping position where the living tissue is grasped by the jaws 9, and the position of the fixed portion where the position of the living tissue remains unchanged in the traction state.

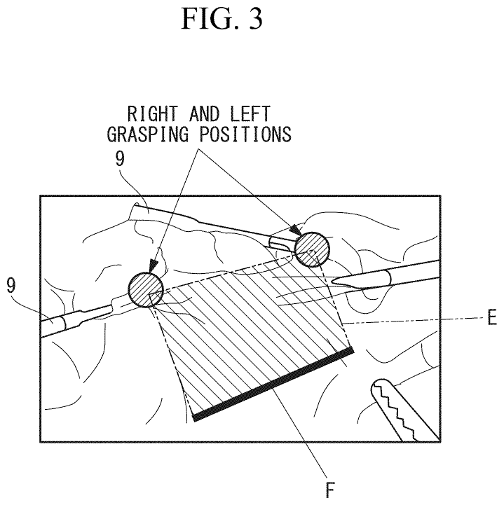

Using the first model adjusted by machine learning, the evaluation unit 15 recognizes the position where the tissue is grasped by the jaws 9 and the tissue structure, such as the fixed position of the living tissue, in the current endoscopic image based on the measurement results given by the measurement unit 13. In machine learning of the first model, for example, as shown in FIG. 3, a plurality of previous endoscopic images accompanied by the jaws 9, the positions to be grasped by the jaws 9, and the tissue structure are used as teacher data. Hereinafter, models adjusted by machine learning are simply referred to as “model,” “first model,” and “second model”. For example, a convolutional neural network (CNN) or deep neural network (DNN) is used as the models, first models and second models.

The evaluation unit 15 sets an evaluation region E on the current endoscopic image of the traction operation being performed based on the recognized grasping position and tissue structure. For example, as shown in FIG. 3, the evaluation unit 15 may set, as the evaluation region E, a polygonal region that includes the fixed line (fixed portion of the membrane tissue) F where the position of the membrane tissue remains unchanged in the traction state recognized from the current endoscopic image and at least two tissue grasp positions in the living tissue for the jaws 9.

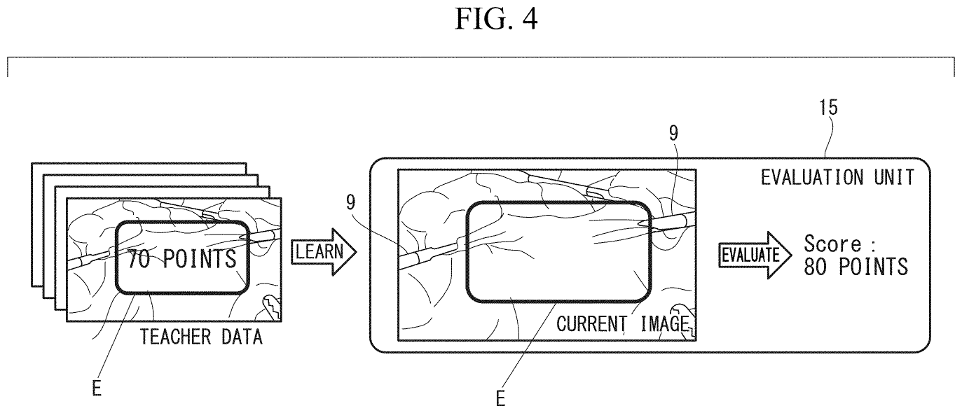

As shown in FIG. 4, for example, after the image of the evaluation region E is cut out of the current endoscopic image, the evaluation unit 15 evaluates the state of traction of the living tissue by the jaws 9 in the evaluation region E, using the second model. As shown in FIG. 4, in the machine learning of the second model, for example, a plurality of previous endoscopic images with scores of the tension of the living tissue being pulled by the jaws 9 are used as teacher data. The evaluation is represented, for example, by a score. Note that the teacher data shown in FIG. 4 is a conceptual image. In reality, the scores should not necessarily be shown on the previous endoscopic images: the scores may be prepared as text data or other data associated with the endoscopic image data, such as jpeg. The same is true for both the learning of binary values such as “OK: suitable” and the learning of arrows indicating the traction direction in the descriptions of the teacher data in the subsequent embodiments and modifications, and these information should not necessarily be depicted in the image. “A plurality of previous endoscopic images accompanied by” herein do not necessarily mean “depicted on the image” but also includes association in a separate file, for example.

The presentation unit 17 constructs traction assistance information that indicates the evaluation of the traction state of the living tissue based on the evaluation results given by the evaluation unit 15. The traction assistance information includes, for example, a score of the evaluation to be displayed on the monitor 7 and a position where the score will be displayed. The presentation unit 17 also adds the constructed traction assistance information to the current endoscopic image.

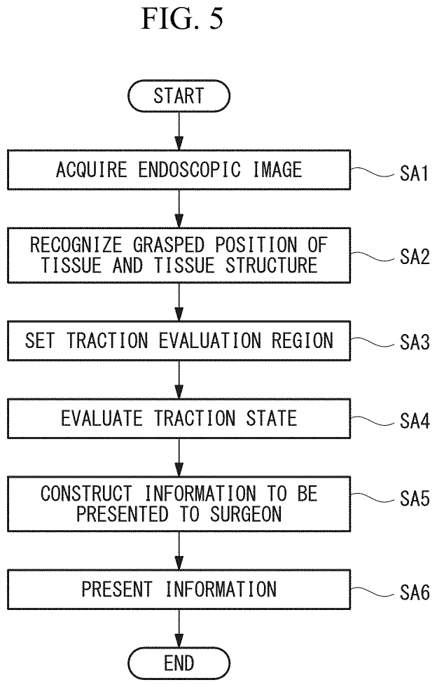

The action of the endoscope system 1, manipulation assistance method, and manipulation assistance program in the aforementioned configuration will now be explained with reference to the flowchart of FIG. 5 and FIG. 2.

To assist manipulation by a surgeon using the endoscope system 1, manipulation assistance method, and manipulation assistance program, the living tissue being pulled by the jaws 9 is first imaged by the endoscope 3. The endoscopic image of the living tissue acquired from the endoscope 3 is captured by the first I/O device 11 into the control device 5 (Step SA1).

Next, the measurement unit 13 measures the feature values related to the grasp of the living tissue in the captured current endoscopic image. The evaluation unit 15 then uses the first model to recognize the tissue structure, such as the positions of tissue to be grasped by the jaws 9 and the fixed position of the living tissue in the current endoscopic image, based on the measurement results given by the measurement unit 13 (Step SA2).

Next, the evaluation unit 15 sets the evaluation region E on the current endoscopic image based on the recognized tissue grasping position and tissue structure (Step SA3).

Afterwards, the image of the evaluation region E is cut out of the current endoscopic image by the evaluation unit 15, and the second model is then used to evaluate the state of traction of the living tissue by the jaws 9 in the evaluation region E based on a score (Step SA4).

Next, the presentation unit 17 constructs, for example, the text “Score: 80 points” or the like as traction assistance information based on the evaluation results given by the evaluation unit 15 (Step SA5). The constructed traction assistance information is added to the current endoscopic image and then sent to the monitor 7 via the second I/O device 19. As a result, the text “Score: 80 points” indicating the evaluation of the traction state of the living tissue is presented on the monitor 7 together with the current endoscopic image (Step SA6).

As explained above, with the endoscope system 1, manipulation assistance method, and manipulation assistance program, when an endoscopic image of a living tissue being pulled by the jaws 9 is acquired by the endoscope 3, the measurement unit 13, evaluation unit 15, and presentation unit 17 of the control device 5 derive traction assistance information on the traction of the living tissue by the jaws 9. The traction assistance information is then displayed on the monitor 7 in association with the current endoscopic image. This allows the surgeon to perform the traction operation according to the traction assistance information while viewing the current endoscopic image, thereby improving the stability of the manipulation operation and equalizing the manipulation independently of the surgeon's experience.

In addition, the evaluation results for the traction operation contribute to unification of the evaluation criteria for the traction operation, which would conventionally depend on the experience of each surgeon. Consequently, the surgeon pulls the living tissue according to the traction assistance information, which reduces variations in manipulation operations by the surgeon and further equalizes the manipulation operation.

This embodiment can be modified as follows.

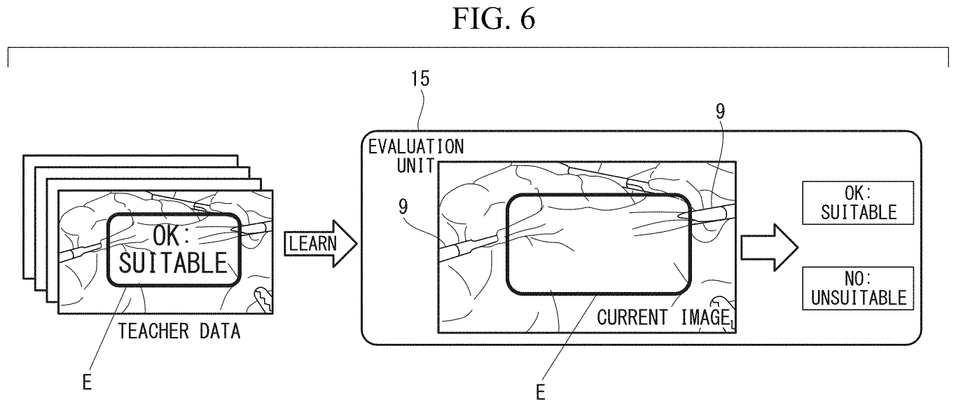

In a first modification, as shown in FIG. 6, the evaluation unit 15 may, for example, evaluate the traction state of the living tissue in the evaluation region E in a binary value indicating suitability or unsuitability. The evaluation unit 15 may, for example, evaluate the suitability or unsuitability based on a predetermined threshold by quantifying the tension of the living tissue being pulled to determine the traction state of the living tissue. In machine learning, a plurality of previous endoscopic images accompanied by a binary evaluation value representing suitability or unsuitability of the state of the tension of the pulled living tissue may be used as the teacher data. The binary evaluation value may be the text “OK: suitable” that means that the tension of the living tissue is suitable or the text “NO: unsuitable” that means that it is not suitable. The presentation unit 17 may construct the text “OK: suitable” or “NO: unsuitable” as traction assistance information.

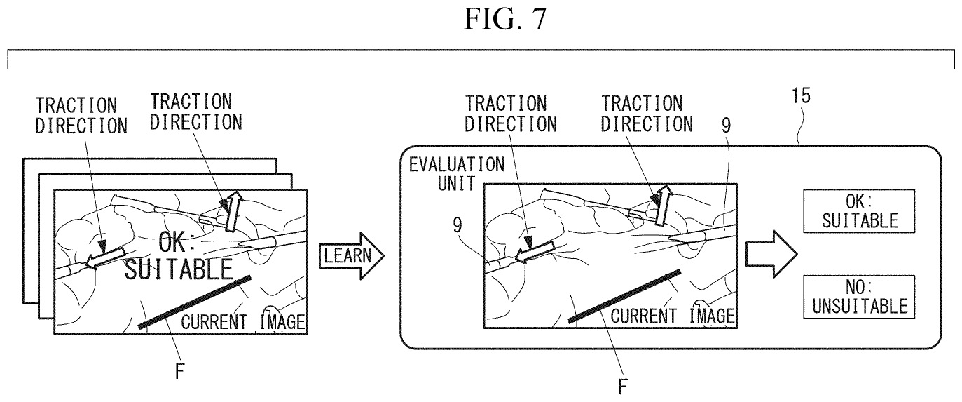

In this modification, as shown in FIG. 7, the evaluation unit 15 may, for example, evaluate the traction direction of the living tissue on a suitability/unsuitability basis based on the predetermined threshold. In machine learning, a plurality of previous endoscopic images accompanied by the fixed line F indicating the position of the fixed portion of the membrane tissue, arrows indicating the traction directions of the living tissue being pulled, and a binary evaluation value representing suitability or unsuitability for the traction direction may be used as teacher data. The binary evaluation value may be, for example, the text “OK: suitable” that means that the traction direction for the living tissue is suitable or the text “NO: unsuitable” that means that it is not suitable. The presentation unit 17 may construct arrows indicating the traction directions the fixed line F of the membrane tissue as well as the text “OK: suitable” or “NO: unsuitable” as traction assistance information.

In the second modification, the evaluation unit 15 may evaluate the traction state from changes in the feature values of the living tissue in the evaluation region E. Examples of the feature values of the living tissue include the color of the surface of the living tissue. When the living tissue is pulled, the color of the living tissue becomes lighter as the thickness of the living tissue becomes thinner. Examples of the feature values also include linear components of capillaries. When the living tissue is pulled, the linear components of capillaries in the living tissue increase. Examples of the feature values also include the sparse density of capillaries. When the living tissue is pulled, the density of capillaries in the living tissue increases. Examples of the feature values also include the isotropy of the fibers of the connective tissue. When the living tissue is pulled, the fibers of the connective tissue are aligned in one direction. Examples of the feature values also include the distance between the grasping portions of the living tissue grasped by a plurality of jaws 9. For example, the suitable traction condition is that the rate of change of the distance between the portions grasped by one pair of jaws 9 and the other pair of jaws 9 in the evaluation region before and after traction is about 1.2 to 1.4 times. Instead of the rate of change of the distance between the grasping portions of the living tissue by the a plurality of jaws 9 before and after traction, the rate of change of the distance between the a plurality of jaws 9 grasping the living tissue before and after traction may be adopted.

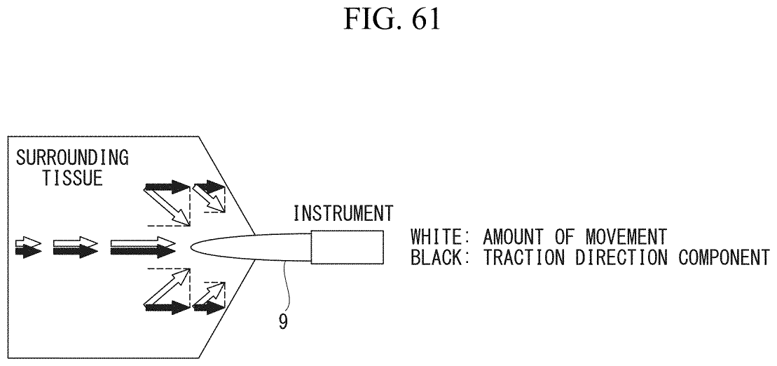

Examples of the feature values also include the distribution of the amount of movement of the living tissue. This is effective in situations where living tissue is pulled by a single instrument, for example, in energetic treatment situations. Traction increases variations in the distribution of the amount of movement of the living tissue around the traction point, i.e., the surrounding tissue. For example, as shown in FIG. 61, when living tissue is pulled by an instrument such as jaws 9, the surrounding tissue near the instrument moves to the same extent as the amount of movement of the instrument, whereas the amount of movement of the surrounding tissue decreases with distance from the instrument. In addition, the distribution of the amount of movement of the surrounding tissue varies greatly in the direction of traction.

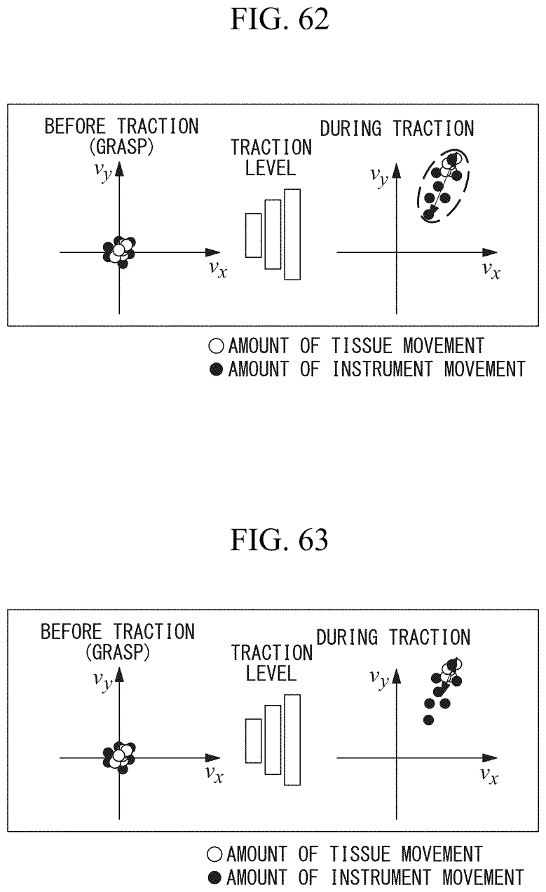

Therefore, for example, as shown in FIG. 62, variations in the first principal component of the principal component analysis of the surrounding tissue may be used as a quantification index. Also, as shown in FIG. 63, the distance between the centers of gravity of the distributions of the amounts of movement of the surrounding tissue and the instrument may be used as a quantification index. However, since the amount of movement is variable, the quantitative evaluation of tension is made by accumulating variations in the first principal component or the distance between the centers of gravity of the surrounding tissue or instrument as a quantification index. In FIGS. 62 and 63, vₓ shows the x-direction of the amount of movement of the surrounding tissue and instrument in the endoscopic image, and v_{y} shows the y-direction of the amount of movement of the surrounding tissue and instrument in the endoscopic image. The amount of movement of the living tissue in the image is the sum of the amount of movement of the living tissue itself and the amount of movement caused by the movement of the endoscope 3. The surgical assistance program may evaluate only the amount of movement of the living tissue itself by compensating the amount of movement caused by the movement of the endoscope 3 from the amount of movement of the living tissue in the image. The amount of movement of the living tissue in the image may be compensated taking into consideration the scale of the subject living tissue by, for example, using information on the instrument.

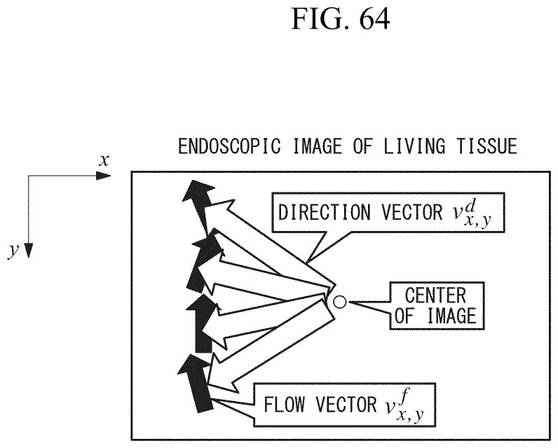

Examples of the feature values also include an evaluation of the amount of movement of the living tissue, taking into account the direction of displacement. This is effective in situations where living tissue is deployed by traction using a plurality of instruments. For example, as shown in FIG. 64, the amount of movement of the surrounding tissue with respect to the reference point of traction may be used as an evaluation index for traction or relaxation. In this case, for example, the center of the image in the evaluation region E may be used as the reference point for traction. However, since the amount of movement is variable, the quantitative evaluation of tension is made by accumulating the amounts of movement of the surrounding tissue with respect to the reference point of traction as a quantification index. For example, as shown in the equation below, it may be quantified by adding up the inner products of the vector between the center of the image, i.e., direction vector v^{d}, and the vector of the amount of movement of each pixel, i.e., flow vector of in the evaluation region E.

$$a = \frac{1}{n} {\sum }_{x , y} {v}_{x , y}^{f} \cdot {v}_{x , y}^{d}$$

Here, x is the x-direction in the endoscopic image and y is the y-direction in the endoscopic image.

The surface morphology of the structure may also be used as a feature value, for example, means of using vascular density, bright spots, spatial frequency, color histogram shape, and the like.

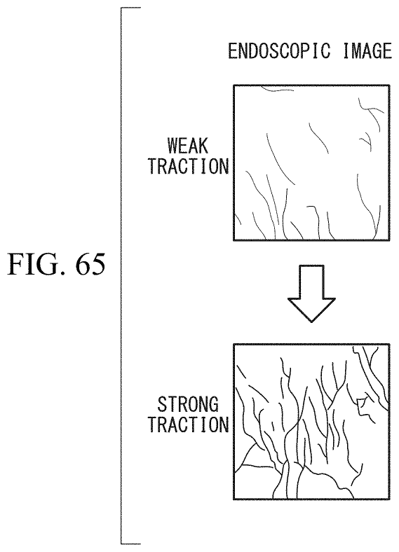

For example, as shown in FIG. 65, the stronger the traction force, the greater the exposure of small blood vessels in the superficial layer of the surrounding tissue. For this reason, the amount of change in the density of blood vessels may be acquired by extracting blood vessels from the endoscopic image of the surrounding tissue. In this case, for example, after binarizing the image of the evaluation region E from which the blood vessels have been extracted, the sum of the brightness values of the image may be used as a quantification index.

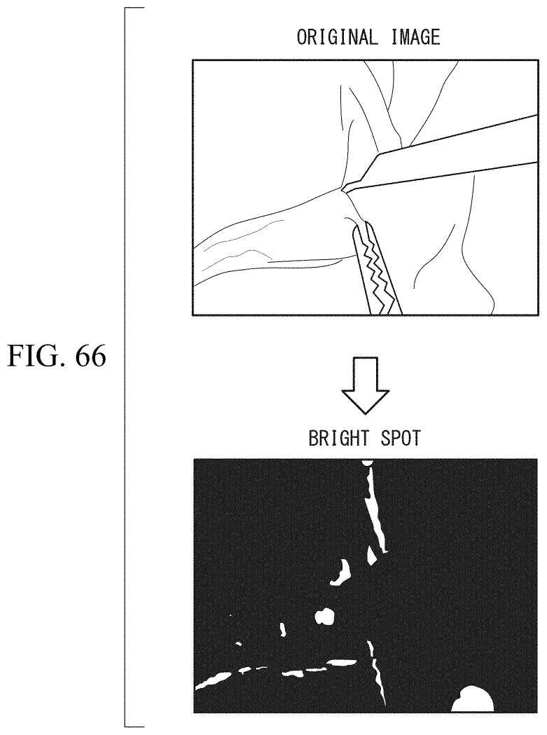

As shown in FIG. 66, when tension is applied to the surrounding tissue by traction, the number of bright spots increases due to specular reflection, for example. For this reason, the cumulative area of bright spots in the image of the evaluation region E may be used as a quantification index.

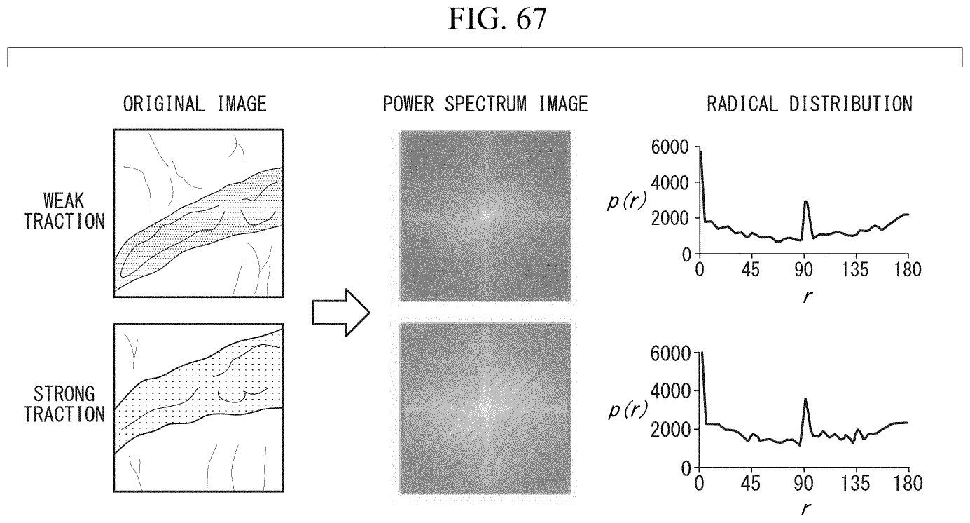

For example, a change in spatial frequency of the surrounding tissue due to the application of tension may be evaluated by spatial frequency analysis of the endoscopic image. In this case, for example, as shown in FIG. 67, the stronger the traction force, the more high-frequency components in the image, so the radial distribution of the power spectrum may be used as a quantitative evaluation index.

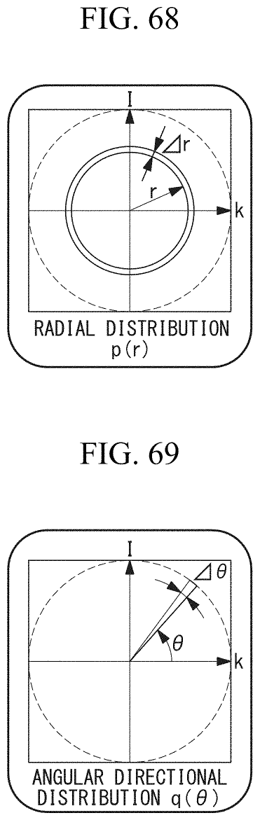

For example, the radial distribution p(γ) shown in FIG. 68 determines the average of the power spectrum p for each certain distance width Δγ for distance γ, according to the following equation.

$$p ( r ) = \frac{1}{n} {\sum }_{{r}^{′} = r - Δ r / 2}^{r + Δ r / 2} {\sum }_{θ = 0}^{{θ}_{max}} p ( θ , {r}^{′} )$$

Here, θ_ₘₐₓ is the maximum value to be measured in relation to the angle θ.

On the other hand, the angular directional distribution q(θ), shown for example in FIG. 69, determines the average of the power spectrum p for each certain measurement width Δθ in terms of the angle θ, according to the following equation.

$$q ( θ ) = \frac{1}{n} {\sum }_{{θ}^{′} = θ - Δθ / 2}^{θ + Δθ / 2} {\sum }_{r = 1}^{{r}_{max}} p ( {θ}^{′} , r )$$

where γₘₐₓ is the maximum value to be measured with respect to the distance γ and n is the total number of pixels subject to the sum.

The γ component indicates the surface roughness and smoothness of the elements in the image, and the θ component indicates the directionality of the elements in the image.

As shown in FIG. 67, in the power spectrum image, the frequency is lower toward the center of the image and higher outward from the center. Comparison between the power spectrum image with weak traction and the power spectrum image with strong traction shows that the power spectrum image in with strong traction is wider and fuzzier. This means that the power spectrum image with strong traction force has more high-frequency components. This proves that the spatial frequency of the image changes with the strength of the traction force. For example, the value of the high-frequency region of the radical distribution, i.e., the value of γ in the large region, may be used as a quantitative evaluation index.

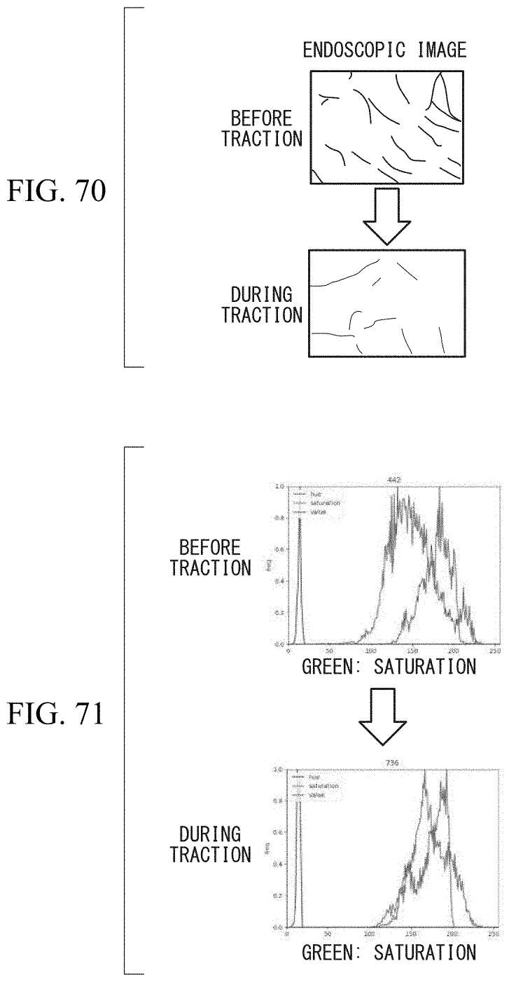

Since traction reduces the shading by eliminating the laxity of the surrounding tissue, as shown in FIG. 70 for example, if the surface shape of the surrounding tissue changes due to traction, as shown in FIG. 71, the shape of the color histogram of the endoscopic image of that surrounding tissue will change. For this reason, a change in the shape of the color histogram of the image of the evaluation region E may be quantified by moment features, e.g., mean, variance, or the shape of the graph such as skewness and kurtosis. An example of a histogram of saturation shown in FIG. 69 shows that the kurtosis is higher at the time of traction than before traction.

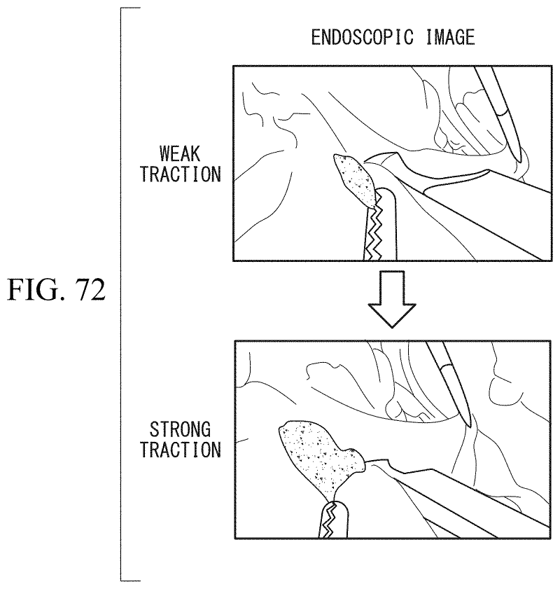

Examples of feature values also include the shape of the edge of the incision region. For example, as shown in FIG. 72, the stronger the traction force, the smaller the curvature of the edge of the incision region in the surrounding tissue. FIG. 72 shows that the stronger the traction force, the smaller the curvature of the edge on the left in the incision region to the viewer of the image. For example, the curvature of the edge of the incision region or the coefficient of curve fitting, such as quadratic function fitting, may be used as a quantification index.

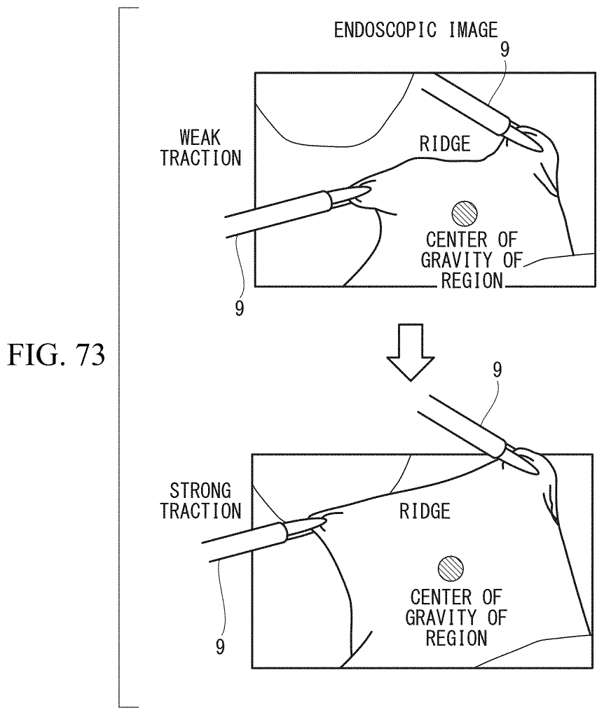

Examples of feature values also include the area or shape of the stretch region surrounded by the grasping positions of the living tissue by a plurality of instruments. The stretch region may be, for example, a polygonal region surrounded by a fixed portion of the membrane tissue where the position of the membrane tissue remains unchanged in the traction state and at least two tissue positions of the living tissue grasped by the jaws 9. The stronger the traction force, the more the living tissue is stretched, so the area, contour, and ridge shape of the stretch region changes with the strength of the traction force, as shown in FIG. 73, for example. A ridge refers to a portion of the contour of the stretch region that connects the 9 jaws. Thus, the area, contour, or ridge shape of the stretch region may be used as a quantification index. For example, the frequency or amplitude of the graph may be used as a traction feature value, graphing the distance between the contour of the stretch region and the center of gravity of the stretch region using polar coordinates. The curvature of the ridge shape may also be used as a quantification index.

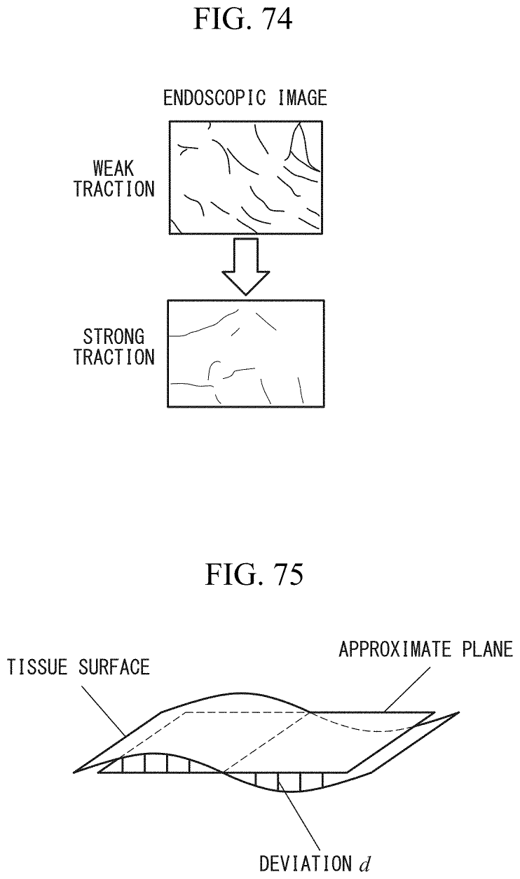

Examples of feature values also include the surface texture of the surrounding tissue. Surface texture can include various levels of undulation and irregularity, ranging from surface undulations to the particle level. One example is the surface irregularity of the surrounding tissue. If the surface irregularities of the surrounding tissue are flattened by tensioning, the color variation of the surface of the surrounding tissue is reduced. For example, as shown in FIG. 74, the stronger the traction force, the smaller the color variation. For example, color deviation, depth deviation or pixel homogeneity may be used to evaluate variation. For example, as shown in FIG. 75 and the equation below, the surface irregularity of the surrounding tissue can be quantified by plane fitting the approximate plane and calculating the sum of the deviation between the surface of the surrounding tissue and the approximate plane.

$$sum\_dev = \vert {\sum }_{x , y} {d}_{x , y} \vert$$

For example, the gray-level co-occurrence matrix (GLOM) can be used to calculate the angular second moment (ASM) which is an index of pixel homogeneity.

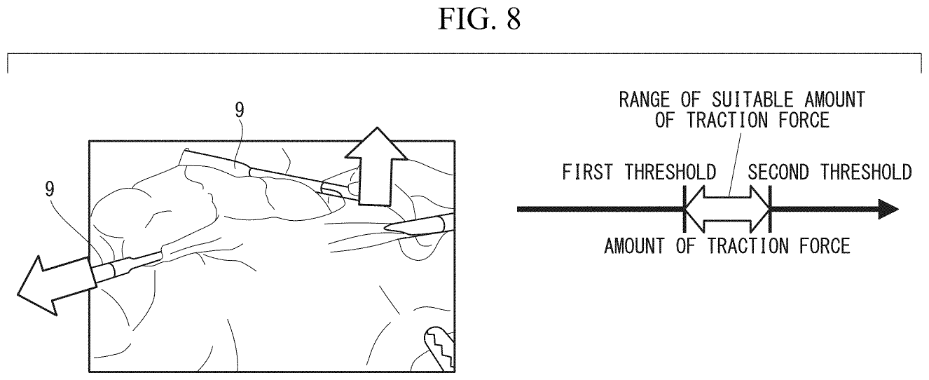

In the third modification, the jaws 9 may include a sensor (not shown in the drawings) to measure the traction force. As shown in FIG. 8, the evaluation unit 15 may, for example, evaluate the traction state of the living tissue using the first and second thresholds for the values measured by the sensor of the jaws 9. The lower limit of the suitable traction force range may be used as the first threshold and the upper limit as the second threshold. If the value measured by the sensor, i.e., the traction force of the jaws 9, is less than the first threshold (e.g., 4 N.), the incision operation cannot be performed comfortably due to weak tension in the living tissue. If the measured value is greater than the second threshold (e.g., 6 N.), damage to the living tissue or slipping of the grasp occurs.

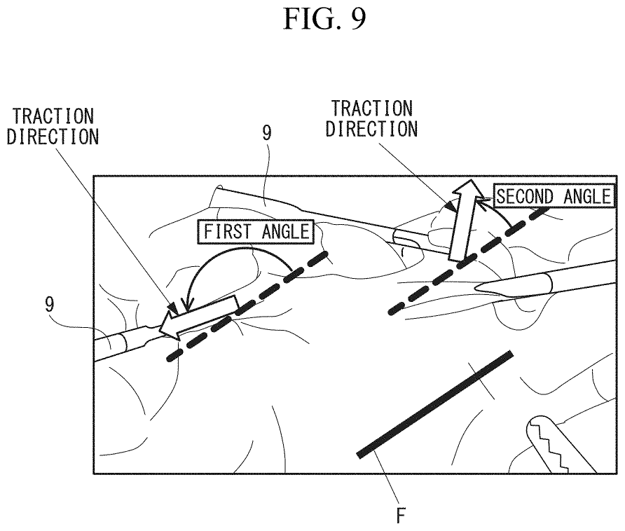

In the fourth modification, as shown in FIG. 9, the measurement unit 13 may measure, as feature values of the current endoscopic image, the movement vectors of the two jaws 9, the first angle between the movement vector of one pair of jaws 9 and the fixed line F of the tissue being pulled, and the second angle between the movement vector of the other pair of jaws 9 and the fixed line F of the tissue being pulled. The evaluation unit 15 may evaluate the traction state of the living tissue based on the movement vectors of the two jaws 9, the first angle, and the second angle measured by the measurement unit 13, without using machine learning. When both the first and second angles are in the range of 0 to 180° and the first angle—the second angle ≥0°, the surgical field can generally be formed. In this case, the first angle should be obtuse and the second angle should be acute. More suitable ranges for the angles are defined depending on the incision site and the surgical scene.

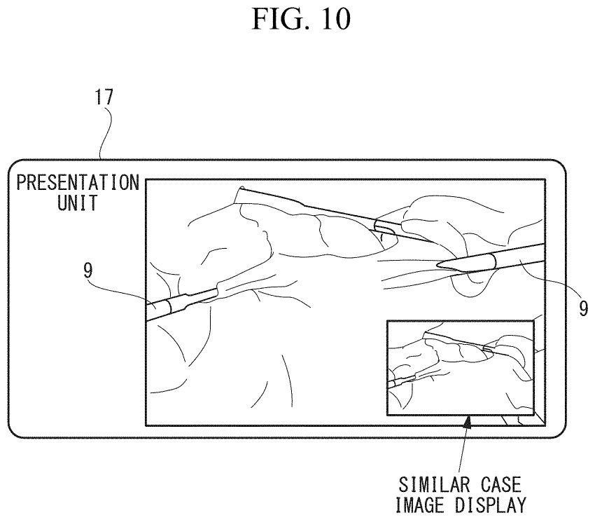

In the fifth modification, the evaluation unit 15 may use machine learning to evaluate the shape features of the living tissue in a traction state. Based on the evaluation by the evaluation unit 15, the presentation unit 17 may select endoscopic images of the living tissue in a traction state with similar shape features, from an image library (not shown in the drawings). As shown in FIG. 10, the selected endoscopic images of similar cases may be added to the current endoscopic image as traction assistance information. As a result, the endoscopic images of similar cases are superimposed on the current endoscopic image on the monitor 7. The machine learning may use teacher data with tissue information indicating the location of the fixed portion of the membrane tissue.

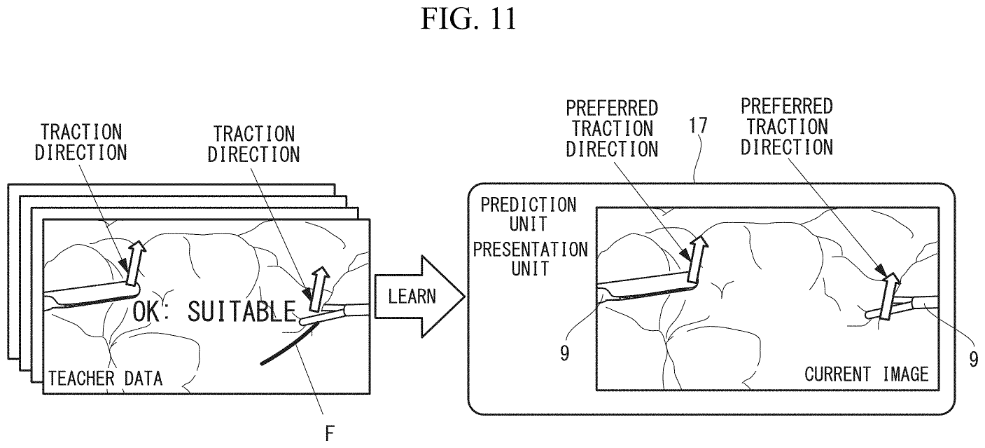

In the sixth modification, the control device 5 may include a prediction unit (not shown in the diagrams) instead of the evaluation unit 15. The prediction unit may set the evaluation region E by the same method as the evaluation unit 15. The prediction unit may also use a model to predict traction directions in which a preferred traction state of the living tissue in the evaluation region E can be achieved. In machine learning, for example, as shown in FIG. 11, a plurality of previous endoscopic images with the fixed line F of the membrane tissue, arrows indicating the directions of traction by the jaws 9, and the text “OK: suitable” or “No: unsuitable,” which are binary values to evaluate the suitability of traction directions, may be used as teacher data. In this case, the presentation unit 17 may construct traction assistance information, such as arrows indicating preferred traction directions, based on the prediction results given by the prediction unit. As a result, arrows indicating preferred traction directions are presented on the current endoscopic image on the monitor 7.

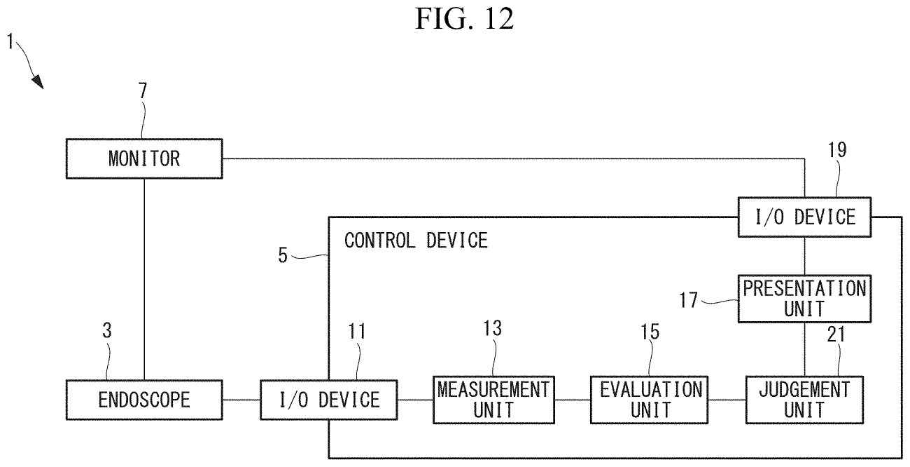

In the seventh modification, as shown in FIG. 12, the control device 5 may include a judgement unit 21 that judges whether or not additional traction operations are required, based on the evaluation results given by the evaluation unit 15, as well as the first I/O device 11, measurement unit 13, evaluation unit 15, presentation unit 17, and the second I/O device 19. The judgement unit 21 may be composed of the processor 14.

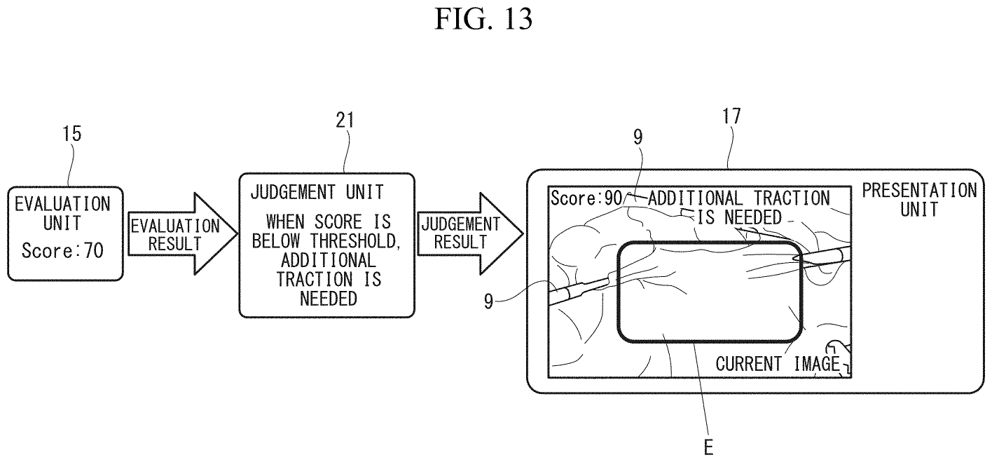

For example, as shown in FIG. 13, in a configuration where the evaluation by the evaluation unit 15 is represented by a score, the judgement unit 21 may judge that an additional traction operation is necessary when the score is below a predetermined threshold. If the judgement unit 21 judges that an additional traction operation is necessary, the presentation unit 17 may construct traction assistance information, such as “additional traction required” text indicating the need for an additional traction operation. As a result, the “additional traction required” text that urges the surgeon to perform an additional traction operation is presented in the current endoscopic image on the monitor 7.

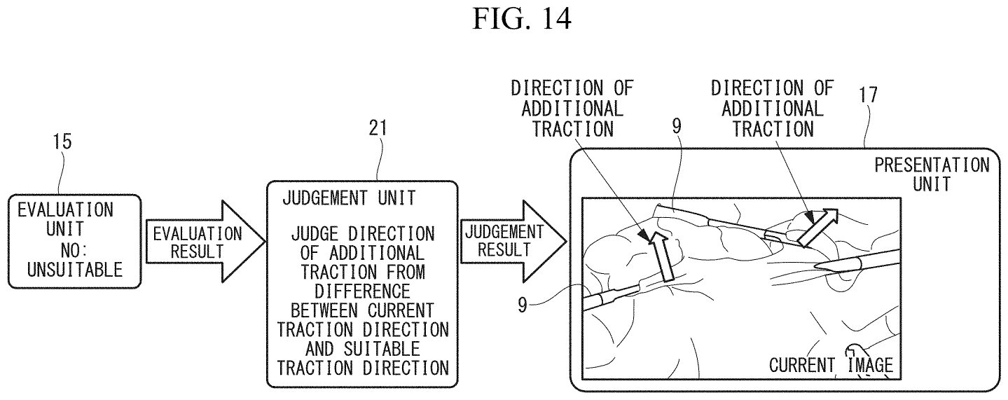

As shown in FIG. 14, for example, in a configuration where the evaluation by the evaluation unit 15 is represented by a binary value, when the evaluation result is “NO: unsuitable,” the judgement unit 21 may judge an additional traction direction based on the difference between the current traction direction and a suitable traction direction. If the additional traction direction is judged by the judgement unit 21, traction assistance information such as an arrow indicating the additional traction direction may be constructed by the presentation unit 17. Hence, an arrow indicating the additional traction direction is presented in the current endoscopic image on the monitor 7.

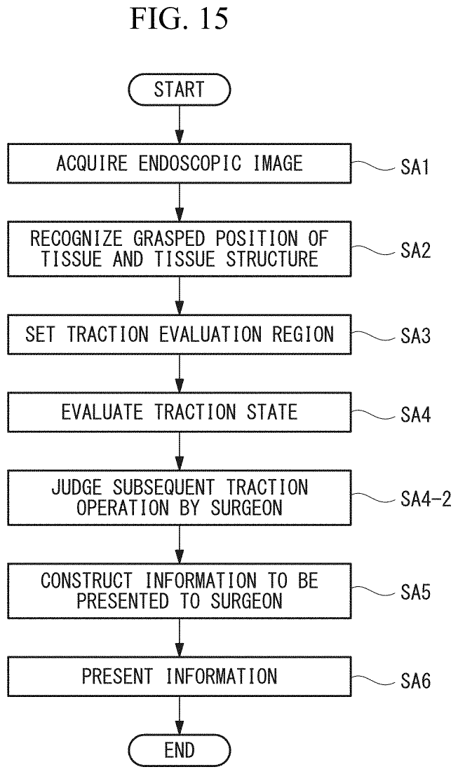

In the eighth modification, the control device 5 may include the prediction unit according to the sixth modification and the judgement unit 21 according to the seventh modification, as well as the measurement unit 13, evaluation unit 15, and presentation unit 17. The judgement unit 21 may determine the next traction operation based on at least one of the prediction result given by the prediction unit and the evaluation result given by the evaluation unit 15 (Step SA4-2), as shown in the flowchart of FIG. 15. The presentation unit 17 may construct traction assistance information, such as characters indicating the next traction operation, based on the determination by the judgement unit 21 (Step SA5). In this modification, the control device 5 may include a drive control unit that drives the manipulator, which is not shown in the drawings, based on the next traction operation determined by the judgement unit 21. The drive control unit may also be composed of the processor 14.

### Second Embodiment

An endoscope system, manipulation assistance method, and manipulation assistance program according to the second embodiment of the present invention will be described below with reference to the accompanying drawings.

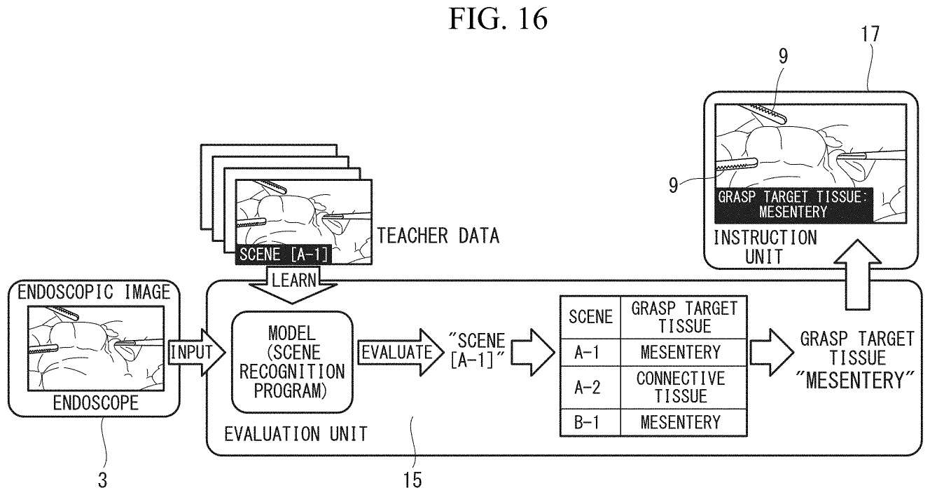

As shown in FIG. 16, for example, the endoscope system 1 of this embodiment differs from the one in the first embodiment in that it outputs grasp assistance information related to the grasping operation of living tissue by jaws 9.

In the description of this embodiment, the parts whose configurations are the same as in the endoscope system 1 of the first embodiment described above are denoted by the same reference numerals thereas, and their descriptions will be omitted.

The manipulation assistance program causes the control device 5 to perform an acquisition step of acquiring an image of living tissue being grasped by a jaw (instrument) 9 or the like; a derivation step of deriving traction assistance information related to the living tissue grasp operation by the jaws 9 based on the acquired living tissue image; and a display step of displaying the derived grasp assistance information in association with the living tissue image. As for the manipulation assistance program, each step of the manipulation assistance method, which will be described below, is executed by the control device 5.

The measurement unit 13 measures feature values related to a surgical scene in the current endoscopic image that has been captured.

The evaluation unit 15 uses a model to recognize the current surgical scene based on the measurement results given by the measurement unit 13. In machine learning, a plurality of previous endoscopic images accompanied by the names of the respective surgical scenes are used as teacher data. The names of the surgical scenes are, for example, “Scene {A-1},” “Scene {A-2},” “Scene {B-1},” and the like.

The evaluation unit 15 reads a living tissue associated with the recognized surgical scene from a database in which a plurality of surgical scene names are associated with the names of target living tissues to be grasped, and thus determines the read living tissue as a grasp target tissue.

The presentation unit 17 constructs grasp assistance information indicating the grasp target tissue, based on the grasp target tissue that has been determined by the evaluation unit 15, and then adds the constructed grasp assistance information to the current endoscopic image. The grasp assistance information contains, for example, the name of the grasp target tissue to be displayed on the monitor 7 and a position in the display where the tissue name will be displayed.

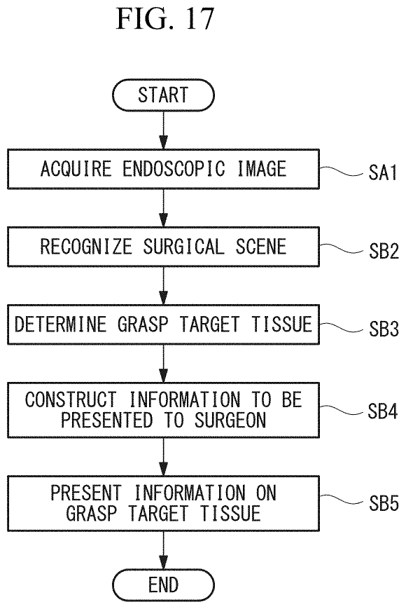

The action of the endoscope system 1, manipulation assistance method, and manipulation assistance program in the aforementioned configuration will now be explained with reference to the flowchart of FIG. 17 and FIG. 16.

To assist manipulation by a surgeon using the endoscope system 1, the manipulation assistance method, and the manipulation assistance program according to this embodiment, when an endoscopic image of the living tissue acquired from the endoscope 3 is captured by the control device 5 (Step SA1), the measurement unit 13 measures feature values related to a surgical scene in the captured current endoscopic image.

Next, the evaluation unit 15 uses a model to recognize the currently performed surgical scene as, for example, “scene {A-1}” based on the measurement results given by the measurement unit 13 (Step SB2). The evaluation unit 15 then determines the “mesentery” as a grasp target tissue by reading the “mesentery” associated with “scene {A-1}” from the database (Step SB3).

Subsequently, the presentation unit 17 constructs, for example, the text “Grasp target tissue: mesentery” to be displayed in the lower left of the endoscopic image as grasp assistance information (Step SB4), and the constructed grasp assistance information is added to the current endoscopic image. As a result, the text “Grasp target tissue: mesentery” is presented in the lower left of the current endoscopic image on the monitor 7 (Step SB5).

As explained above, with the endoscope system 1, manipulation assistance method, and manipulation assistance program according to this embodiment, the measurement unit 13, evaluation unit 15, and presentation unit 17 of the control device 5 determine the grasp target living tissue on behalf of the surgeon according to the surgical scene, and the information indicating the name of the determined grasp target tissue is presented in the current endoscopic image. This allows the surgeon to correctly grasp the necessary living tissue and to properly perform the subsequent tissue traction. Besides, it eliminates the need for grasping living tissue that does not need to be grasped and reduces the risk of damaging the living tissue.

This embodiment can be modified in the following manner.

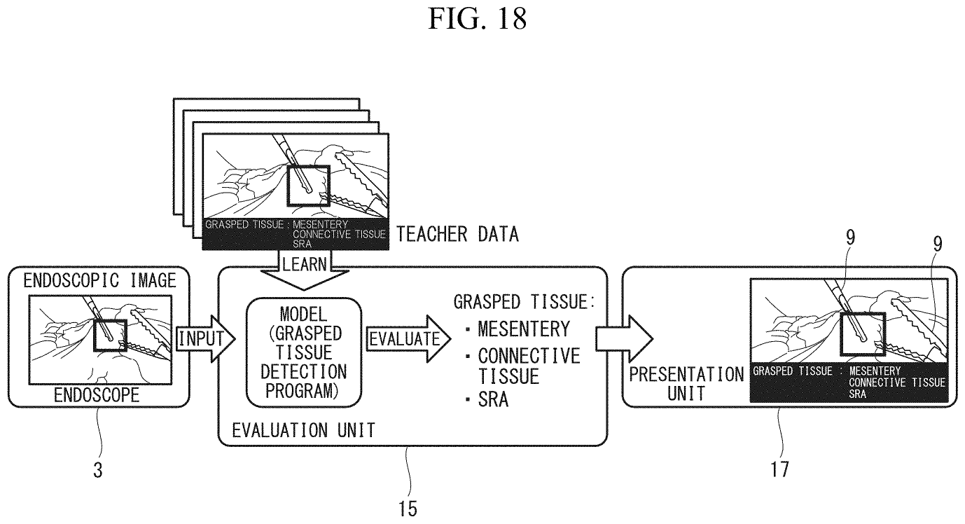

As shown in FIG. 18, for example, in the first modification, the evaluation unit 15 may use a model to detect and evaluate the living tissue currently grasped by the jaws 9. In machine learning, a plurality of previous endoscopic images accompanied by the name of the grasped tissue, for example, “Grasped tissue: mesentery, connective tissue, SRA (superior rectal artery)” may be used as teacher data.

The presentation unit 17 may construct grasp assistance information containing the name of the currently grasped tissue, the position where the tissue name will be displayed, and the like based on the currently grasped living tissues, such as mesentery, connective tissue, and SRA, detected by the evaluation unit 15. As a result, for example, the text “Grasped tissue: mesentery, connective tissue, SRA” is presented in the lower left of the current endoscopic image on the monitor 7.

According to this modification, the surgeon can correctly recognize the currently grasped living tissue based on the grasp assistance information presented in the current endoscopic image. This prevents the surgeon from misrecognizing and reduces the risk of damage to the living tissue.

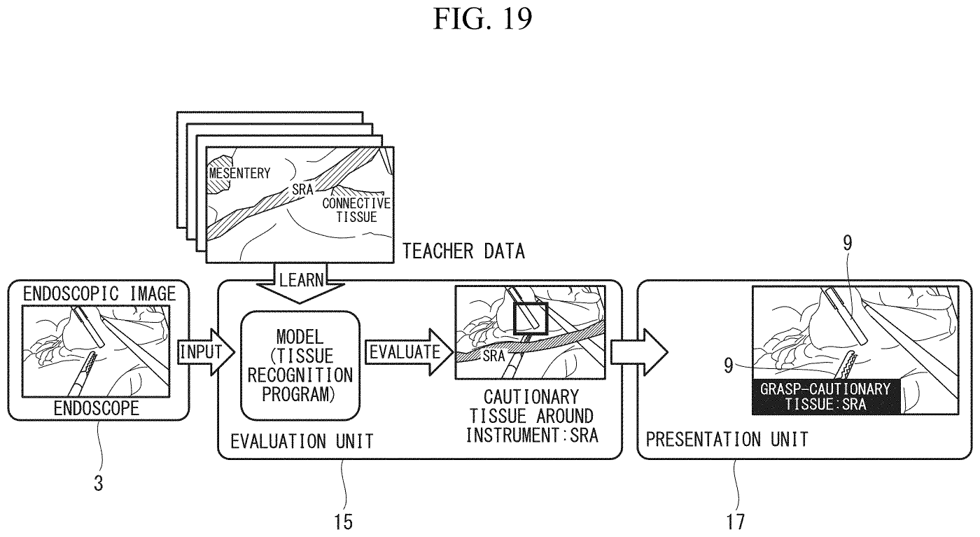

In the second modification, as shown in FIG. 19, for example, the evaluation unit 15 may use a model to detect and evaluate grasp-cautionary tissues that are present around the tip of the jaws 9 and require caution during grasping. In machine learning, a plurality of previous endoscopic images accompanied by tissue region information indicating the region of each living tissue may be used as teacher data. In the tissue region information, regions of each tissue in the endoscopic image may be painted, for example, with different colors and each paint may be denoted by the corresponding tissue name.

The presentation unit 17 may construct grasp assistance information containing the name of a grasp-cautionary tissue and the position where the name of the tissue will be displayed, for example, based on the grasp-cautionary tissue detected by the evaluation unit 15. As a result, for example, the text “Grasp-cautionary tissue: SRA” is presented in the lower left of the current endoscopic image on the monitor 7. Cautionary tissue information indicating the grasp-cautionary tissue may be further presented in the current endoscopic image. In the cautionary tissue information, the regions of the grasp-cautionary tissue in the current endoscopic image may be painted, and each paint may be denoted by the corresponding tissue name.

According to this modification, the surgeon can exercise caution to tissues requiring caution when performing the grasp operation. This prevents incorrect grasp and reduces the risk of tissue damage.

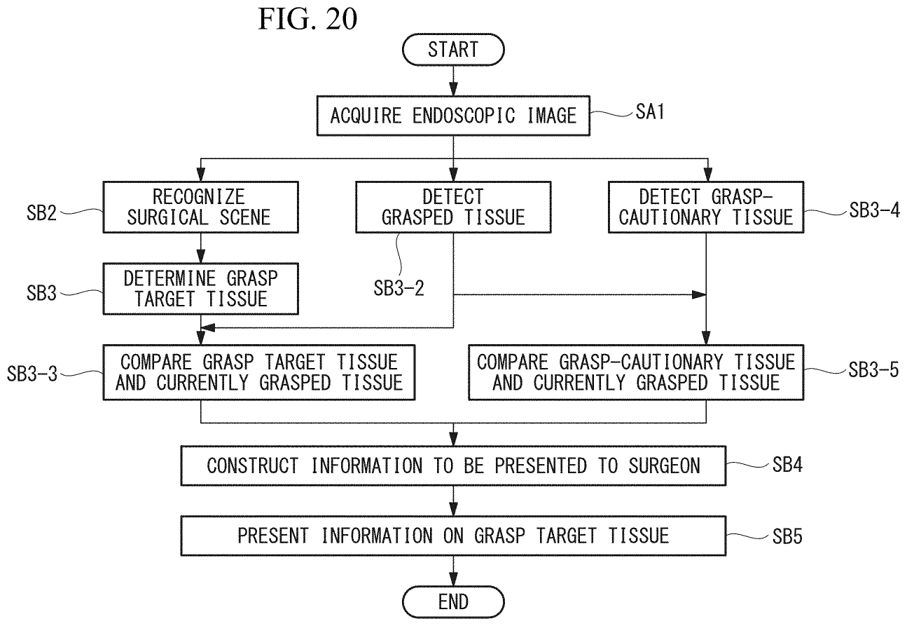

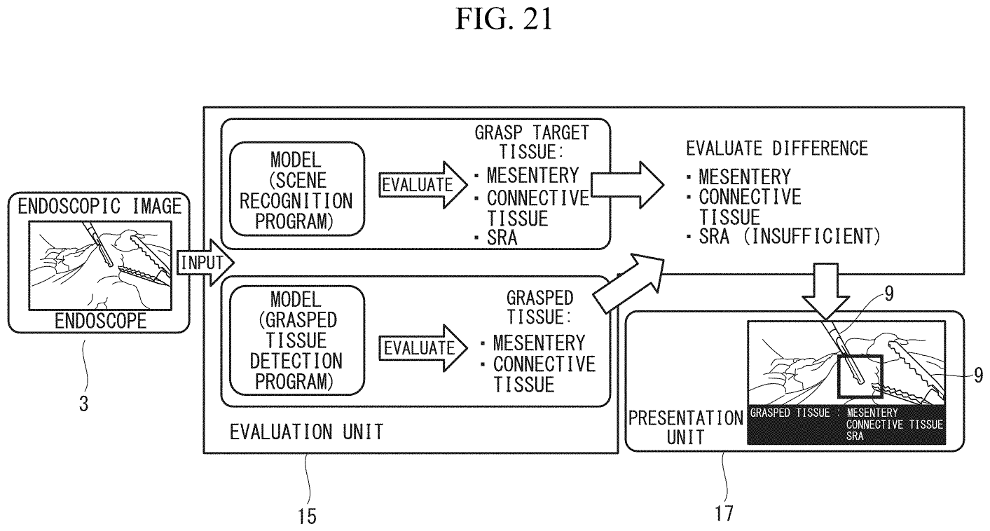

In the third modification, as shown in the flowchart of FIG. 20, for example, the evaluation unit 15 may determine a grasp target tissue (Step SB3), detect and evaluate the currently grasped living tissue (Step SB3-2), and then further evaluate the difference between the grasp target tissue and the currently grasped tissue (Step SB3-3). For example, as shown in FIG. 21, if there are three grasp target tissues which are mesentery, connective tissue, and SRA, while there are two currently grasped living tissues which are mesentery and connective tissue, the evaluation unit 15 evaluates that the SRA is not currently grasped.

For a grasp target tissue, if there is a living tissue that is not currently grasped according to the evaluation results given by the evaluation unit 15, the presentation unit 17 may construct grasp assistance information containing the name of the living tissue that is not currently grasped, a position where that name will be displayed, and the like (Step SB4). As a result, the text “Grasped tissue: mesentery, connective tissue” is presented in the lower left of the current endoscopic image on the monitor 7, and the text “SRA” indicating the tissue that is not currently grasped is presented in a different color or the like so that it can be put in contrast with these tissue names (Step SB5).

According to this modification, the surgeon can correctly grasp suitable living tissue according to the scene properly.

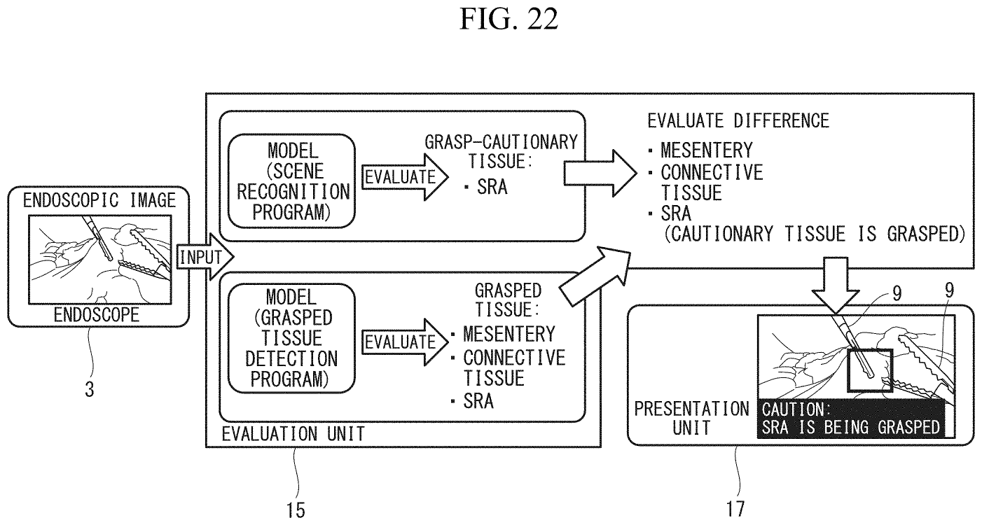

In the fourth modification, as shown in the flowchart of FIG. 20, for example, the evaluation unit 15 may detect the grasp-cautionary tissue that is around the tip of the jaws 9 (Step SB3-4) and detect and evaluate the currently grasped living tissue (Step SB3-2). The evaluation unit 15 may then further evaluate whether or not the grasp-cautionary tissue is being grasped, by checking the grasp-cautionary tissue against the currently grasped tissue (Step SB3-5). For example, as shown in FIG. 22, if the grasp-cautionary tissue is the SRA, while the currently grasped tissues are the mesentery, connective tissue, and SRA, the SRA of the grasp-cautionary tissue is evaluated as being grasped.

If there is a grasp-cautionary tissue is currently grasped according to the evaluation results given by the evaluation unit 15, the presentation unit 17 may construct grasp assistance information containing the name of the grasp-cautionary tissue that is currently grasped, a position where that information will be displayed, and the like. As a result, the text “Caution: SRA being grasped” is presented in the lower left of the current endoscopic image on the monitor 7.

According to this modification, the risk of tissue damage can be reduced.

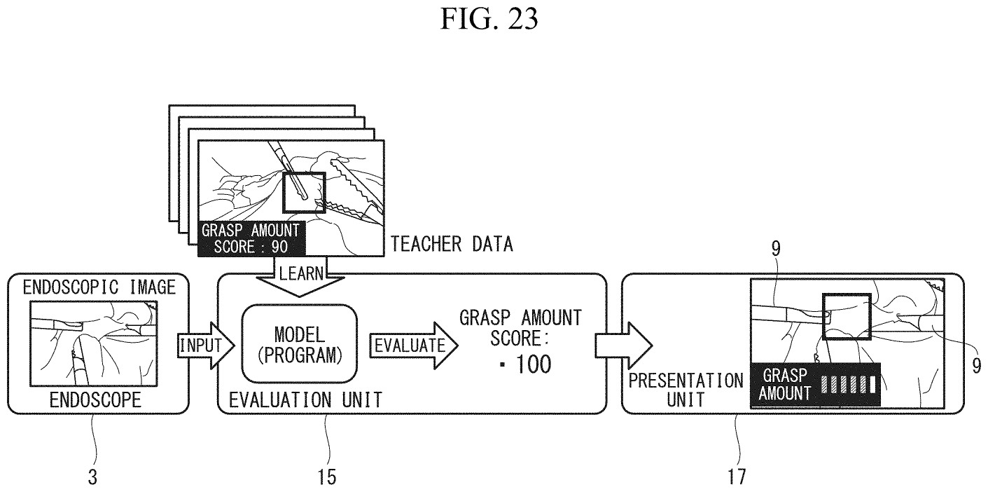

In the fifth modification, as shown in FIG. 23, for example, the evaluation unit 15 may use a model to evaluate the amount of grasp of living tissue currently grasped by the jaws 9 in a score or the like. Since the length of the jaws 9 is known, the amount of grasp by the jaws 9 can be determined based on the length of the jaws 9 exposed on the living tissue in the current endoscopic image. In machine learning, a plurality of previous endoscopic images accompanied by scores indicating the amount of grasp, such as “Score of grasp amount: 90,” may be used as teacher data.

The presentation unit 17 may construct grasp assistance information containing a meter representing the amount of grasp and a position where the meter will be displayed, based on the evaluation results given by the evaluation unit 15. As a result, a meter of the amount of grasp is presented in the lower left of the current endoscopic image on the monitor 7.

According to this modification, the standard for the amount of grasp of living tissue, which would conventionally be dependent on the experience of each surgeon, can be standardized, enabling grasping operations with a suitable amount of grasp.

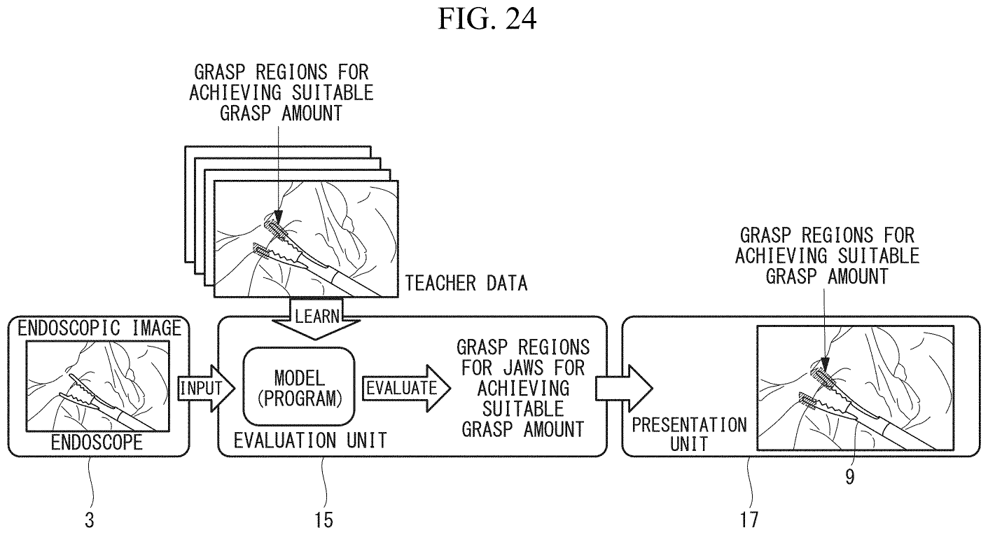

In the sixth modification, as shown in FIG. 24, for example, the evaluation unit 15 may use a model to evaluate the region grasped by the jaws 9 achieving a suitable amount of grasp. In machine learning, a plurality of previous endoscopic images in which the grasped regions achieving the suitable amounts of grasp in the endoscopic images are, for example, painted may be used as teacher data. For the suitable amount of grasp, the lower and upper limits of the amount of grasp for each pair of jaws 9 are preset.

The presentation unit 17 may construct grasp assistance information containing a paint indicating the grasping region where a suitable amount of grasp is achieved, a position where the paint will be displayed, and the like, based on the grasping region evaluated by the evaluation unit 15. As a result, grasp assistance information represented by the paint is presented in the grasping region where the suitable amount of grasp is achieved, in the current endoscopic image on the monitor 7.

According to this modification, the target grasp amount, which would conventionally be dependent on the experience of each surgeon, becomes clear in the endoscopic image, and the grasping operation can be performed with a suitable amount of grasp.

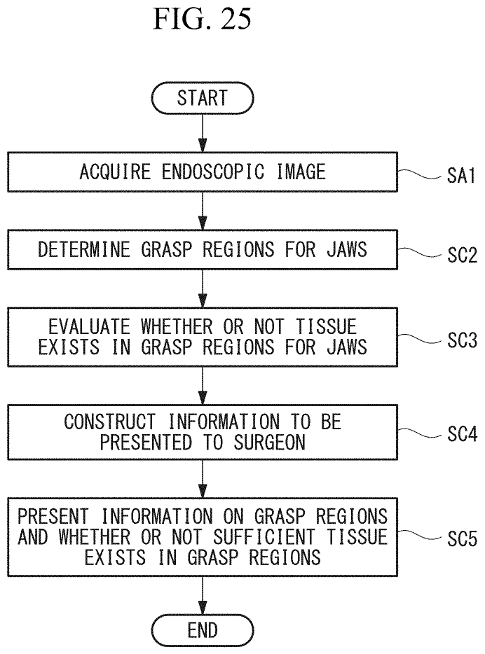

In this modification, the evaluation unit 15 may further evaluate whether the living tissue is positioned in the grasp regions for the jaws 9 without excess or deficiency, by comparing the relationship between the grasp regions for the jaws 9 in which the suitable amount of grasp is achieved and the position of the living tissue. In this case, as shown in the flowchart of FIG. 25, for example, the evaluation unit 15 uses a model to determine the grasp regions for the jaws 9 in which a suitable amount of grasp is achieved (Step SC2). Subsequently, comparing the relationship between the determined grasping region and the position of the living tissue, the evaluation unit 15 evaluates whether or not the living tissue is present in the grasp regions for the jaws 9 (Step SC3).

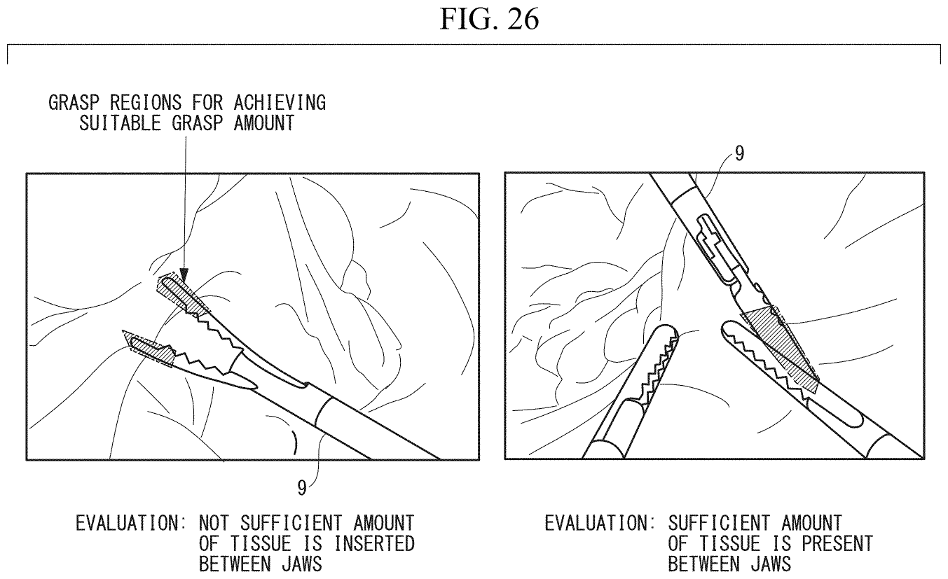

Subsequently, based on the evaluation results given by the evaluation unit 15, the presentation unit 17 constructs grasp assistance information containing a paint or the like indicating the grasping region in which the suitable amount of grasp is achieved, comments on whether or not living tissue exists in that grasping region, and the position where these information will be displayed (Step SC4). As shown in FIG. 26, for example, paint or the like is applied to the grasping region where the suitable amount of grasp is achieved in the current endoscopic image on the monitor 7 and, in association with that endoscopic image, a comment such as “Evaluation: Not sufficient amount of tissue is inserted between the jaws.” or “Evaluation: Sufficient amount of tissue is inserted between the jaws.” is presented.

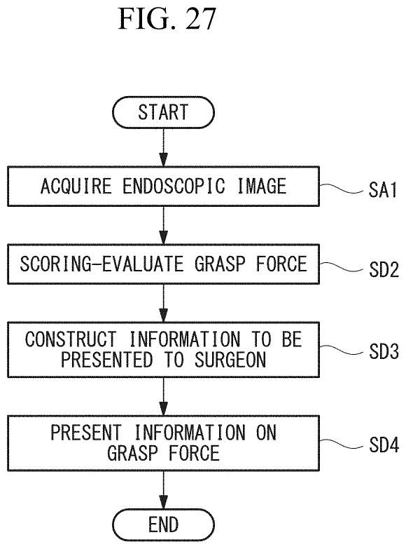

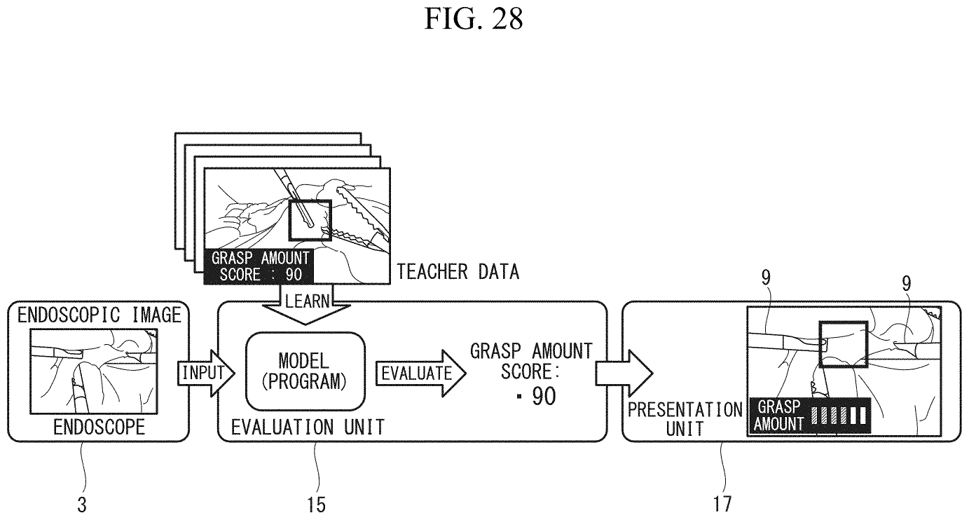

In the seventh modification, as shown in the flowchart of FIG. 27, for example, the evaluation unit 15 may use a model to evaluate the grasping force of the jaws 9 in a score or the like (Step SD2). In machine learning, for example, as shown in FIG. 28, a plurality of previous endoscopic images with a score such as “Grasping force score: 90” indicating the evaluation of the grasping force of the jaws 9 may be used as teacher data.

The presentation unit 17 may construct grasp assistance information containing a meter indicating the current grasping force, a position where the meter will be displayed, and the like based on the evaluation results given by the evaluation unit 15 (Step SD3). As a result, a meter indicating the current grasping force is presented in the lower left of the current endoscopic image on the monitor 7 (Step SD4).

According to this modification, the standard for the amount of grasp of living tissue, which would conventionally be dependent on the experience of each surgeon, can be standardized, enabling grasping operations with a suitable amount of grasp.

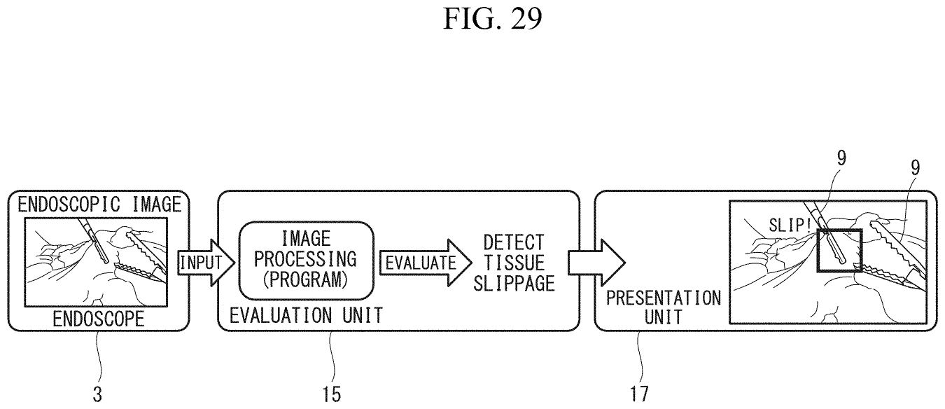

In the eighth modification, as shown in FIG. 29, for example, the evaluation unit 15 may evaluate whether the grasping force of the jaws 9 is insufficient due to slippage of the living tissue, by image processing the current endoscopic image with a predetermined program. If the grasping force of the jaws 9 is insufficient according to the result of the evaluation given by the evaluation unit 15, the presentation unit 17 may construct grasp assistance information including text indicating insufficient grasping force and a position where the text will be displayed. As a result, the text “SLIP!” is presented in the current endoscopic image on the monitor 7 as grasp assistance information.

According to this modification, the surgeon can make adjustments such as increasing the amount of grasping based on the assistance information. This enables grasping with a grasping force that does not cause slippage of the living tissue.

### Third Embodiment

The endoscope system, manipulation assistance method, and manipulation assistance program according to the third embodiment of the present invention will be described below with reference to the accompanying drawings.

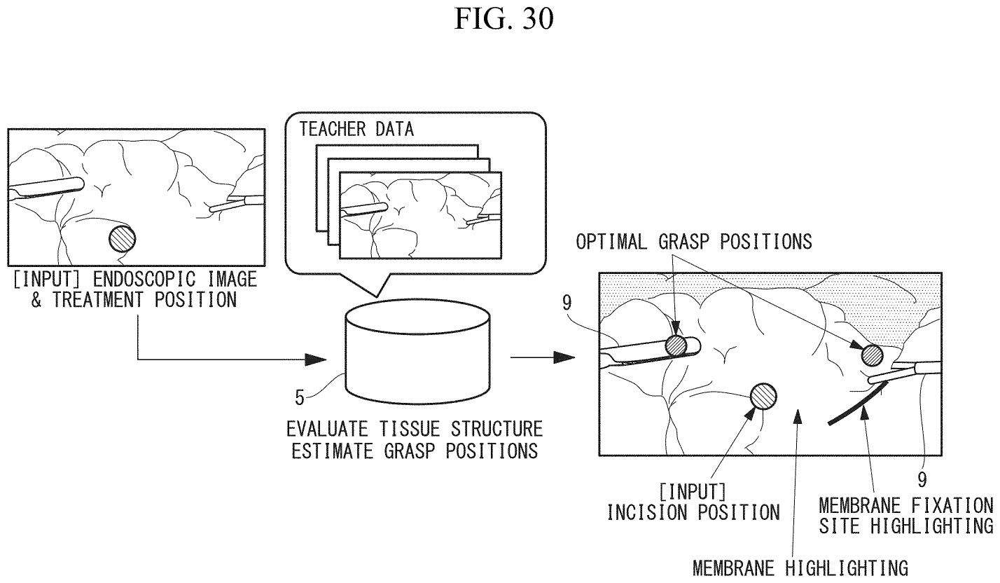

As shown in FIG. 30, for example, the endoscope system 1 according to this embodiment differs from the one in the first and second embodiments in that it outputs grasp assistance information related to positions in the living tissue grasped by the jaws 9.

In the description of this embodiment, the parts whose configurations are the same as in the endoscope system 1 of the first and second embodiments described above are denoted by the same reference numerals thereas, and their descriptions will be omitted. The image acquisition step, derivation step, and display step of the manipulation assistance program are the same as those of the second embodiment. The manipulation assistance program also causes the control device 5 to execute each step of the manipulation assistance method described below.

In this embodiment, grasp assistance information is derived by the control device 5, based on the current endoscopic image acquired by the endoscope 3 and information on the current treatment position. The information on the current treatment position is, for example, input by the surgeon using an input device. Examples of methods of inputting the information include one in which the surgeon gives voice instructions while pressing the jaws 9 against living tissue, one in which the surgeon gives instructions by specific gestures, and one in which the surgeon gives instructions by pressing the touch pen against the screen of the monitor 7. Instead of methods in which the surgeon inputs, the measurement unit 13 may judge the current treatment position based on the current endoscopic image.

The measurement unit 13 measures feature values related to a surgical scene and tissue information in the current endoscopic image that has been captured.

The evaluation unit 15 uses a first model to evaluate the state of the living tissue to be grasped by the jaws 9 based on the current treatment position and the measurement results given by the measurement unit 13. In machine learning, for example, a plurality of previous endoscopic images associated with previous surgical scenes, treatment positions, structure information about the living tissue, and positions grasped by the jaws 9 are used as teacher data. The structure information about the living tissue is, for example, the type of living tissue, the fixed position of the living tissue, the positional relationship, adhesion, and the degree of fat.

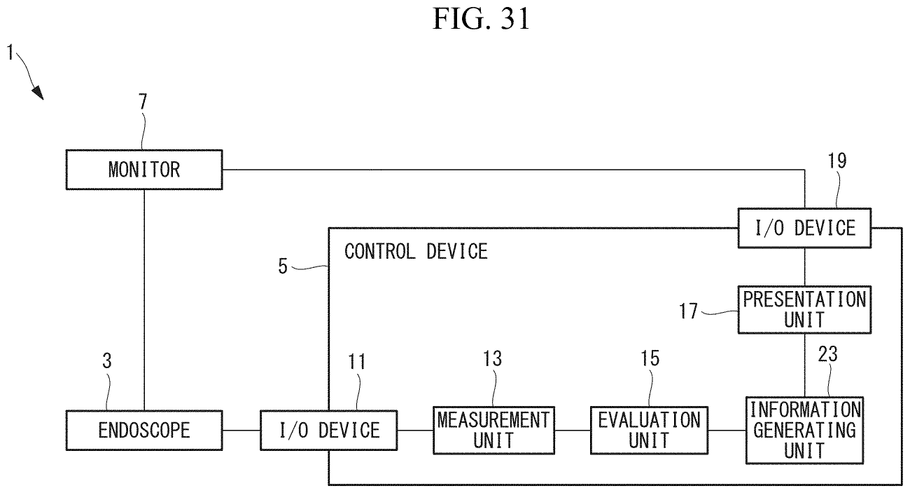

In this embodiment, as shown in FIG. 31, the control device 5 includes an information generating unit 23 composed of the processor 14. The information generating unit 23 uses a second model adjusted by machine learning similar to that for the first model to estimate a suitable grasping position based on the results of the evaluation by the evaluation unit 15.

The presentation unit 17 constructs grasp assistance information indicating the suitable grasping position based on the grasping position that has been estimated by the information generating unit 23, and then adds the constructed grasp assistance information to the current endoscopic image. The grasp assistance information may be, for example, a circle mark or arrow located in the optimal grasping position in the endoscopic image. Other grasp assistance information may include the incision position as a treatment position, membrane highlighting, and membrane fixation site highlighting.

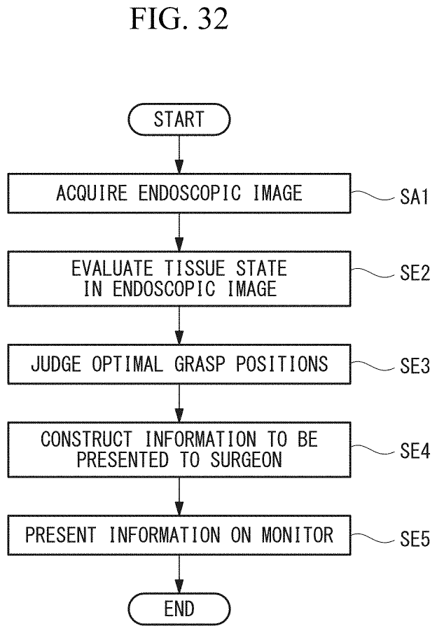

Next, the action of the endoscope system 1, manipulation assistance method, and manipulation assistance program in the aforementioned configuration will be explained with reference to the flowchart of FIG. 32.

To assist manipulation by a surgeon using the endoscope system 1, the manipulation assistance method, and the manipulation assistance program according to this embodiment, when an endoscopic image of the living tissue acquired from the endoscope 3 is captured by the control device 5 (Step SA1), the measurement unit 13 measures feature values related to a surgical scene and tissue information in the captured current endoscopic image. In addition, the current treatment position is input by the surgeon.

Next, the evaluation unit 15 uses the first model to evaluate the state of the living tissue grasped by the jaws 9 based on the measurement results given by the measurement unit 13 and the current treatment position (Step SE2).

Next, the information generating unit 23 uses the second model to estimate a suitable grasping position based on the evaluation results given by the evaluation unit 15 (Step SE3). Subsequently, based on the estimated suitable grasping position, the presentation unit 17 constructs, for example, circled marks to be added to the suitable grasping positions in the endoscopic image (Step SE4), and the constructed circled marks are then added to the current endoscopic image.

Hence, the circled marks indicating optimal positions to be grasped by jaws 9 are presented in the suitable grasping positions in the current endoscopic image on the monitor 7 (Step SE5). In this embodiment, as shown in FIG. 30, the presentation unit 17 further constructs a circled mark indicating the incision position, which is the treatment position, an arrow indicating the membrane highlighting, a line indicating the membrane fixation site highlighting, and the like as grasp assistance information, and the monitor 7 may present these in the current endoscopic image.

As explained above, with the endoscope system 1, manipulation assistance method, and manipulation assistance program according to this embodiment, optimum grasping positions are displayed in the endoscopic image on the monitor 7, which enables matching between recognitions by the surgeon and assistant. Besides, variations among surgeons can be suppressed, which contributes to equalization of manipulation.

This embodiment can be modified in the following manner.

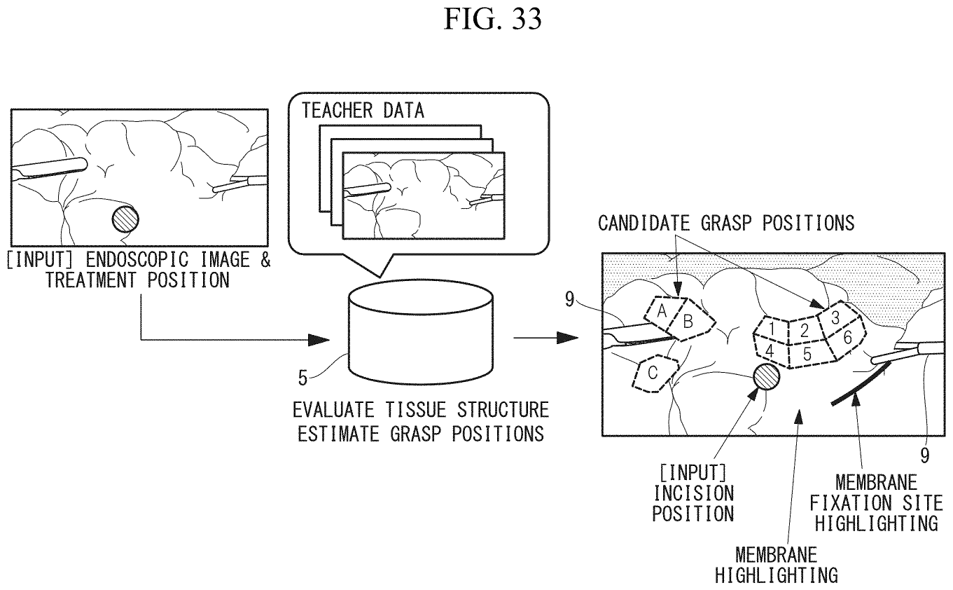

In the first modification, as shown in FIG. 33, for example, the information generating unit 23 may use the second model to estimate a plurality of suitable grasping position candidates based on the results of the evaluation on the state of the living tissue by the evaluation unit 15. Based on the estimated grasping position candidates, the presentation unit 17 may construct grasp assistance information indicating the suitable grasping position candidates. The grasp assistance information may be, for example, a painted region of the suitable grasping position candidates in the endoscopic image, and even also an alphabet or a number to identify each grasping position candidate on each paint.

According to this modification, the optimal grasping position candidates are presented in the current endoscopic image, so that the surgeon or assistant only needs to select a grasping position from the candidates. This enables matching between recognitions by the surgeon and assistant and suppresses variations among surgeons. Grasp assistance information such as paints added to the candidates other than the selected grasping position candidate may be erased from the endoscopic image.

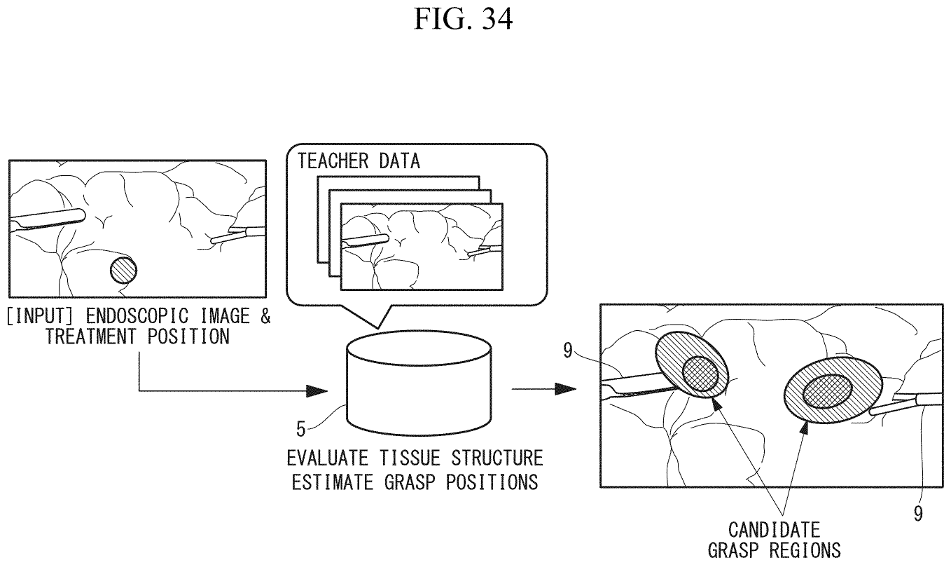

In the second modification, as shown in FIG. 34, for example, the information generating unit 23 may use machine learning to estimate a plurality of suitable grasping position candidates based on the results of the evaluation of the state of the living tissue by the evaluation unit 15, and determine a priority for each grasping position candidate based on the probability that it was grasped properly in previous cases. The presentation unit 17 may also construct grasp assistance information indicating the suitable grasp position candidates and their priorities based on the determination made by the information generating unit 23. The grasp assistance information may, for example, consist of painted areas of suitable grasping position candidates in the endoscopic image, with each paint color shaded according to the priority. For example, the paint color may be made darker for the grasping position candidates that have been grasped properly at a higher probability in previous cases.

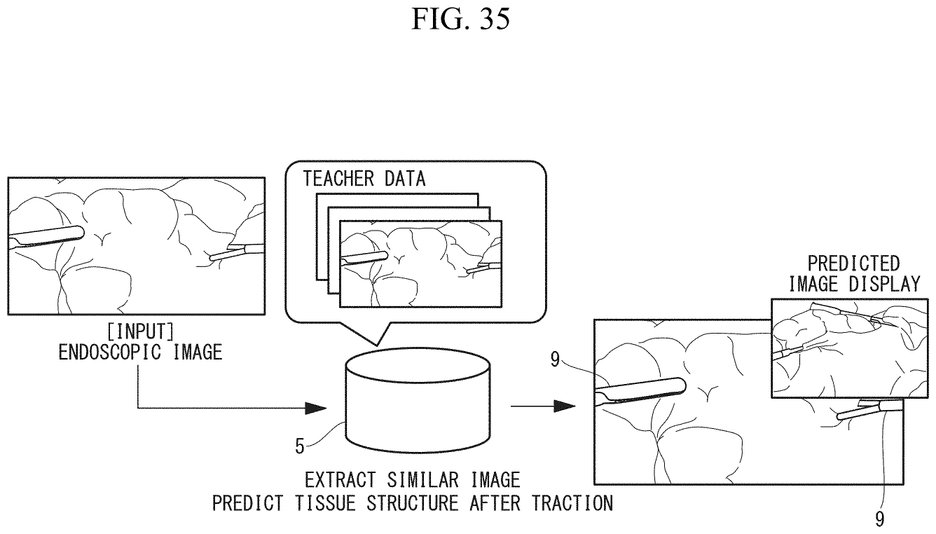

In the third modification, as shown in FIG. 35, for example, the information generating unit 23 may extract images of similar cases and images after traction from a library based on tissue information and then predict the tissue structure brought after tissue traction, using a generative adversarial network (GAN) or the like. The presentation unit 17 may also construct a predictive image of the tissue structure as grasp assistance information based on the tissue structure predicted by the information generating unit 23. The constructed predicted image may be associated with the current endoscopic image and displayed in a sub-window of the monitor 7.

According to this modification, the predicted image after traction, which is useful for selecting grasping positions on the living tissue, is displayed on the monitor 7 together with the current endoscopic image, facilitating the surgeon's judgment on the grasping positions.

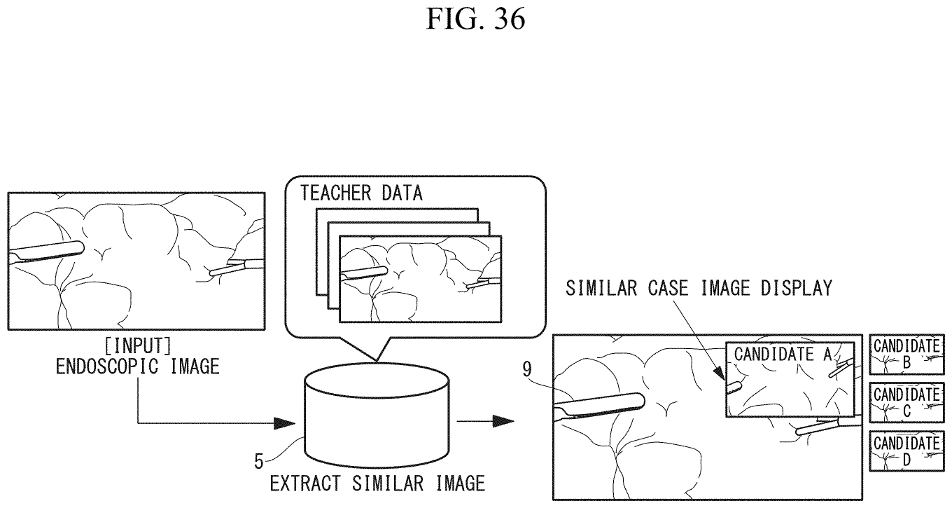

In the fourth modification, as shown in FIG. 36, for example, the information generating unit 23 may extract images of similar cases from the library based on tissue information. Images of similar cases preferably include, for example, previous surgical scenes, treatment positions, tissue structure information, and grasping positions. It is also preferably to extract a plurality of images of similar cases as candidates. The presentation unit 17 may add the images of similar cases extracted by the information generating unit 23 to the current endoscopic image as grasp assistance information. Thus, the images of similar cases may be displayed in a sub-window on the monitor 7 in association with the current endoscopic image. If the displayed image of a similar case does not fit the surgeon's image, the next candidate image of a similar case may be displayed at the surgeon's choice.

According to this modification, images of similar cases that are useful for grasp position selection are displayed on the monitor 7 together with the current endoscopic image, facilitating the surgeon's judgment on the grasp position.

### Fourth Embodiment

The endoscope system, manipulation assistance method, and manipulation assistance program according to the fourth embodiment of the present invention will be described below with reference to the accompanying drawings.

The endoscope system 1, manipulation assistance method, and manipulation assistance program according to this embodiment differ from the ones in the first to third embodiments in that it outputs assistance information related to navigation of grasping operation.

In the description of this embodiment, the parts whose configurations are the same as in the endoscope system 1 of the first to third embodiments described above are denoted by the same reference numerals thereas, and their descriptions will be omitted. The image acquisition step, derivation step, and display step of the manipulation assistance program are the same as those of the first embodiment.

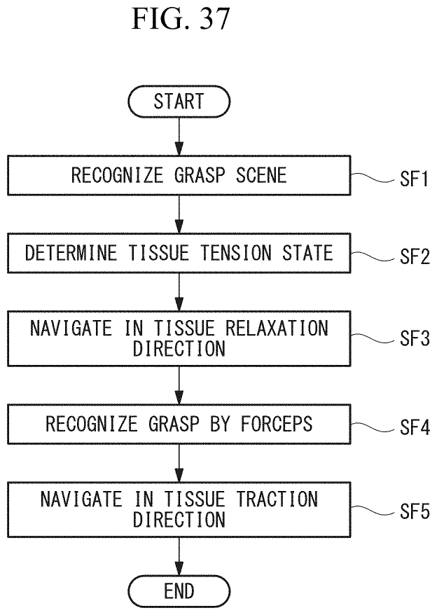

As shown in the flowchart of FIG. 37, the assisted manipulation method according to this embodiment includes a grasping scene recognition step SF1, a tension state determination step SF2, a tissue relaxation navigation step SF3, a grasp recognition step SF4, and a tissue traction navigation step SF5. The manipulation assistance program causes the control device 5 to execute the aforementioned steps SF1, SF2, SF3, SF4, and SF5.

As shown in FIG. 38, for example, the grasping scene recognition step SF1 recognizes that the surgeon is about to grasp living tissue with forceps (instrument) 29. For example, when the surgeon wants to grasp or use the functions of the endoscopy system 1, a predetermined input method is recognized. The recognition method may be recognizing the voice of the surgeon. Alternatively, the recognition method may be recognizing a specific movement pattern of the forceps 29 by the surgeon. The specific movement pattern may be, for example, tapping the part of the living tissue to be grasped by the forceps 29 multiple times or opening and closing the forceps 29. The grasping scene recognition step SF1 is performed by the evaluation unit 15.

The tension state determination step SF2 recognizes the initial tension state of the deployed living tissue for the purpose of restoring the relaxed living tissue to its original state. For example, the initial tension state of the living tissue is recognized using information such as the arrangement pattern of organs and tissues in the endoscopic image and the arrangement and color of capillaries as feature values, and the recognized initial state is remembered. The tension state determination step SF2 is performed by the evaluation unit 15.

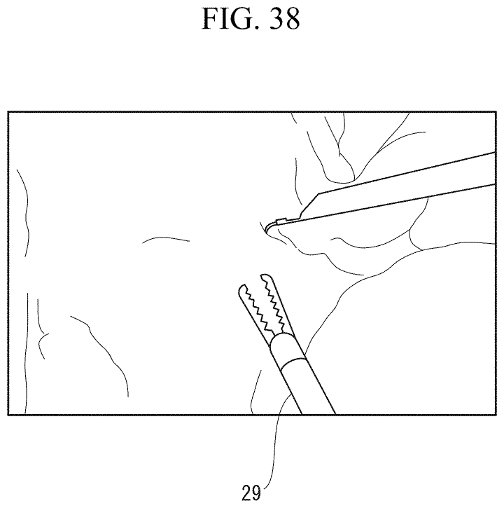

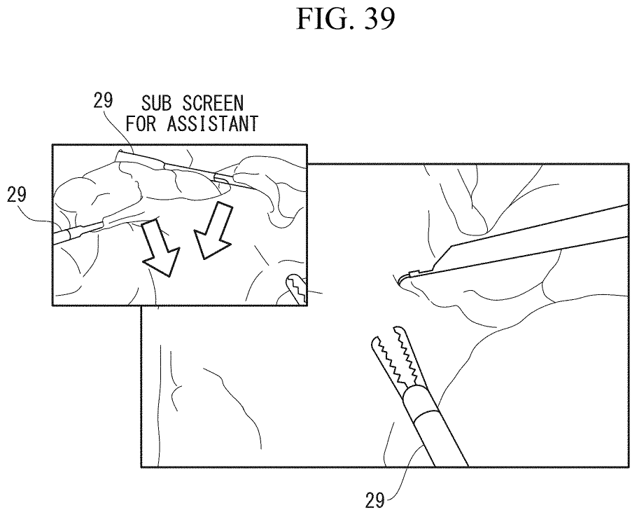

The tissue relaxation navigation step SF3 guides the assistant in the direction of relaxation of the living tissue for the purpose of enabling the surgeon to properly grasp the living tissue. As shown in FIG. 39, the navigation method may be, for example, displaying a sub-screen for the assistant in association with the current endoscopic image on the monitor 7, thereby displaying traction assistance information such as arrows indicating the direction of movement of the assistant's forceps 29 in the sub-screen. The tissue relaxation navigation step SF3 is performed by the evaluation unit 15 and the presentation unit 17.

The evaluation unit 15 performs image analysis after reading the current endoscopic image in real time during navigation. Subsequently, a suitable amount and direction of relaxation are calculated using the capillary morphology and color, and the like as feature values, and the calculated amount and direction of relaxation are reflected in the navigation as needed. Alternatively, the evaluation unit 15 completes the navigation as appropriate by recognizing the voice of the surgeon, the movement pattern of the forceps 29, and the like.

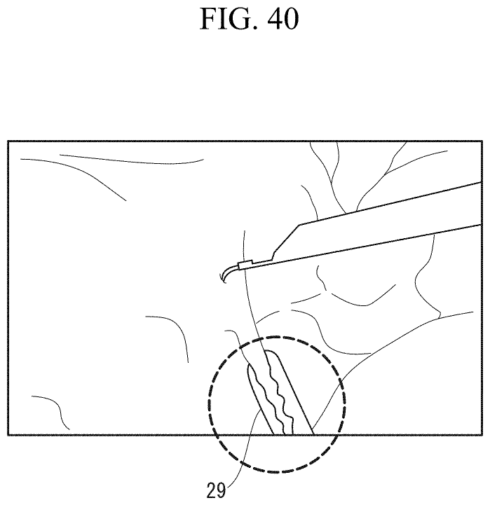

As shown in FIG. 40, for example, the grasp recognition step SF4 recognizes that the surgeon has grasped the living tissue with the forceps 29 by recognizing the surgeon's forceps 29 and the living tissue pinched by the forceps 29 in the endoscopic image. If the image recognition determines that the grasping is not sufficient, from, for example, the protrusion of the living tissue from the forceps 29, this fact may be displayed. In addition, after the fact that the surgeon's forceps 29 are releasing the living tissue is recognized, additional relaxation navigation may be performed. The grasp recognition step SF4 is performed by the evaluation unit 15 and the presentation unit 17.

Tissue traction navigation step SF5 navigates the assistant with the goal of returning to the initial traction state remembered by the tension state determination step SF2. The navigation method is similar to the tissue relaxation navigation step SF3. The tissue traction navigation step SF5 is performed by the evaluation unit 15 and the presentation unit 17.

To assist surgeon's manipulation using the endoscope system 1, manipulation assistance method, and manipulation assistance program, as shown in the flowchart of FIG. 37, first, the evaluation unit 15 recognizes the current grasping scene based on the current endoscopic image acquired by the endoscope 3 (Step SF1), and the tension state of the living tissue is then estimated (Step SF2). Next, based on the information on the estimated tension state, when the surgeon grasps the living tissue with the forceps 29 with one hand, an arrow or the like indicating a direction in which the traction by the assistant is relaxed is presented on the sub-screen of the monitor 7 (Step SF3). Next, the evaluation unit 15 recognizes that the surgeon has grasped the living tissue (Step SF4), and an arrow or the like indicating a direction in which the traction by the assistant is restored is presented on the sub-screen of the monitor 7 (Step SF5).

In laparoscopic surgery, the surgeon and assistant work together to perform the operation safely and efficiently. As an example, the assistant secures the surgical field by pulling and deploying the surrounding tissue with grasping forceps or the like, while the surgeon applies countertraction to the tissue with grasping forceps in one hand while proceeding with incision and separation with an electrocautery scalpel or the like in the other hand. At this time, it is preferable that suitable tension be applied to the surgical field when the assistant pulls the living tissue, in order to accomplish smooth incision with the electrocautery scalpel and to facilitate recognition of the layered structure of the tissues to be separated.

When the surgeon grasps the tissue with the grasping forceps in the left hand, the living tissue is taut due to the traction operation by the assistant, making it difficult to grasp the tissue. As a result, he or she may have to re-grip the tissue many times, or if he or she tries to proceed without grasping the tissue firmly, the incision with the electrocautery scalpel may not be performed smoothly due to lack of suitable countertraction. On the other hand, when an incision and separation operation is performed by the surgeon, the endoscope view is usually zoomed in on the treatment operating part, and in most cases the assistant's forceps are not visible on the screen. Therefore, in principle, the assistant's forceps should not be moved after deployment by the surgeon's forceps manipulation.

With the endoscope system 1, manipulation assistance method, and manipulation assistance program, the living tissue relaxes when the surgeon grasps the living tissue. This allows the grasping operation to be performed more securely. This eliminates the effort to re-grasp the tissue, and the secure grasping enables safe and reliable incisional operations.

This embodiment can be modified in the following manner.

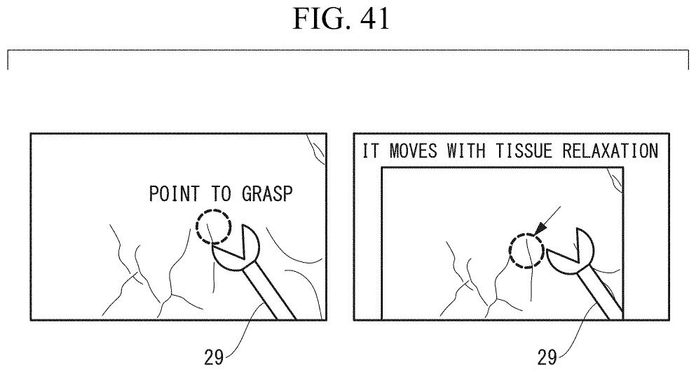

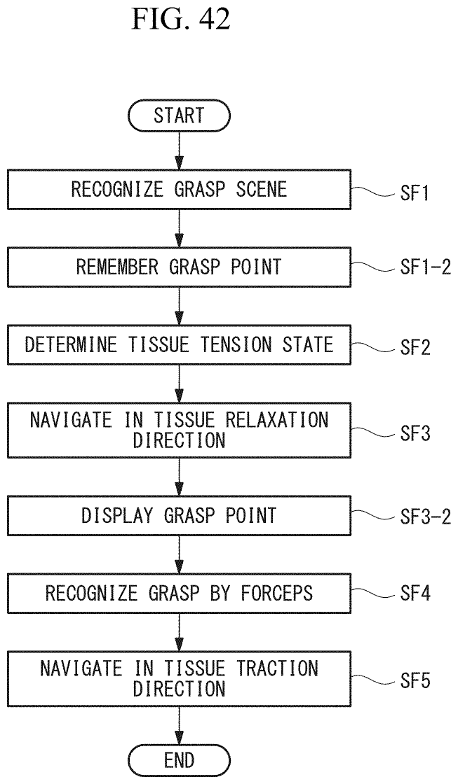

In the first modification, for example, as shown in FIG. 41, the grasp points that the surgeon wants to grasp may be clearly indicated in the endoscopic image, because the points that the surgeon wants to grasp move with the relaxation of the living tissue. This modification includes, for example, the grasp point memory step SF1-2 and the grasp point display step SF3-2, as shown in the flowchart of FIG. 42. The manipulation assistance program causes the control device 5 to perform the steps SF1-2 and SF3-2 described above.

The grasp point memory step SF1-2 remembers the grasp point in the living tissue recognized in the grasping scene recognition step SF1, associating them to feature values such as capillaries in the endoscopic image. The grasp point memory step SF1-2 is performed by the evaluation unit 15.

The tissue relaxation navigation step SF3 preferably track grasp points such as capillaries and other features in real time during relaxation of living tissue.

The grasp point display step SF3-2 estimates the grasp points based on the feature values such as capillaries in the endoscopic image, and then adds marks and the like indicating the estimated grasp points, to the current endoscopic image as grasp assistance information. The grasp point display step SF3-2 is performed by the evaluation unit 15 and the presentation unit 17.

According to this modification, the surgeon can securely grasp the locations that he/she originally wants to grasp.

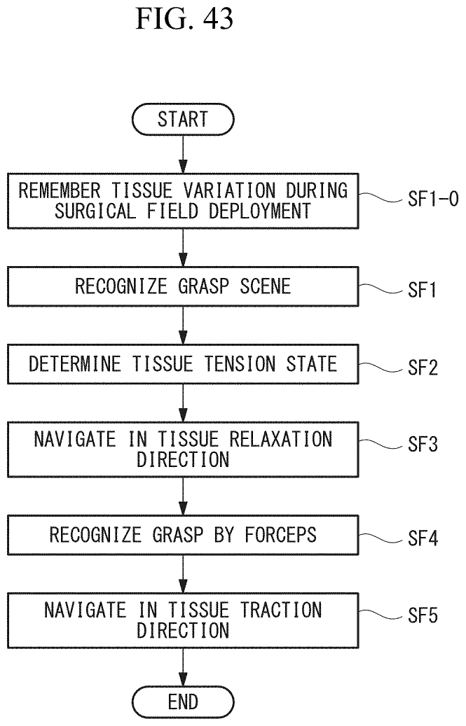

In the second modification, the direction and amount of tissue relaxation during grasping by the surgeon may be estimated in advance based on the tissue variation during the initial surgical field deployment. This modification includes, for example, a tissue variation memory step SF1-0 in which the tissue variation during the surgical field deployment is remembered, as shown in the flowchart of FIG. 43. The manipulation assistance program causes the control device 5 to perform the step SF1-0 described above.

The tissue variation memory step SF1-0 records the amount of traction, direction of traction, and amount of elongation of the living tissue in the current endoscopic image, associating them with feature values such as capillaries. Subsequently, it calculates the amount and direction of relaxation with which the living tissue changes less, from the recorded information. The calculated amount and direction of relaxation are used for tissue relaxation navigation in the tissue relaxation navigation step SF3. The tissue variation memory step SF1-0 is performed by the evaluation unit 15.

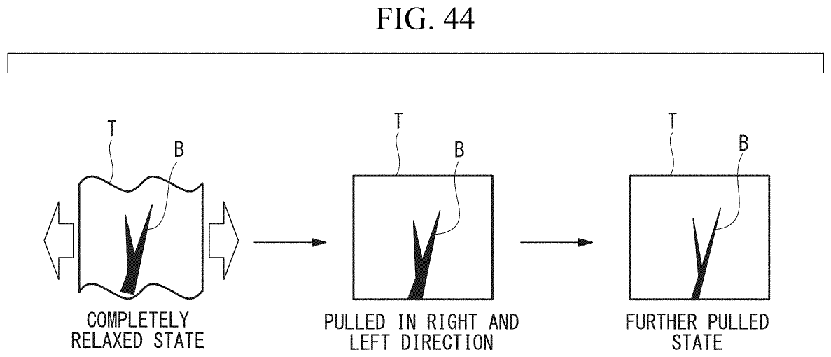

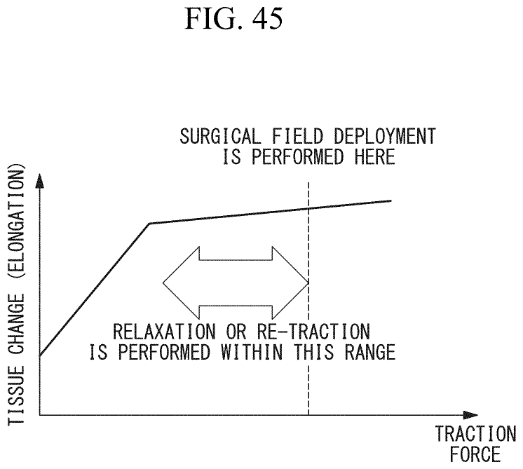

When living tissue is pulled, as shown in FIGS. 44 and 45, up to a certain amount of traction, elongation occurs as the living tissue is deformed, but from a certain level of traction, the changes in the living tissue become smaller. In FIG. 44, the reference T indicates the living tissue and the reference B indicates the capillaries. This modification performs relaxation of the living tissue in the region where the amount of change in the living tissue is small. According to this modification, the amount of tissue movement during tissue relaxation is minimized, so that the surgeon can perform suitable grasping operations without disrupting the surgical field.

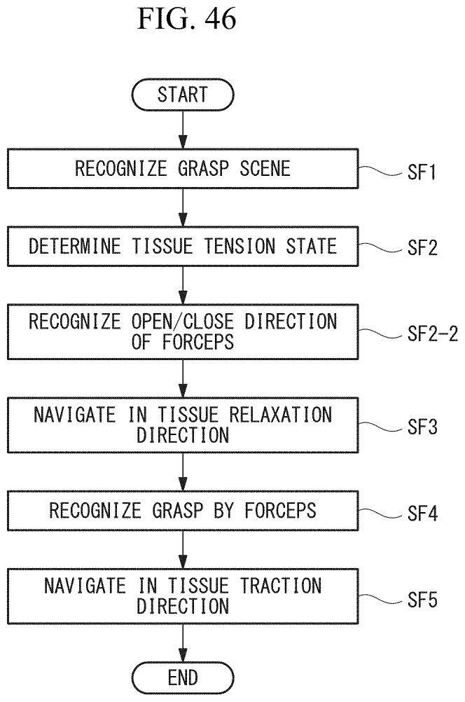

In the third modification, in the case where the assistant uses two forceps 29, traction assistance information instructing to move only one of the forceps 29 during relaxation and re-traction of the living tissue may be presented. As shown in the flowchart of FIG. 46, this modification includes, for example, an open/close direction recognition step SF2-2 in which the open/close direction of the surgeon's forceps 29 is recognized from the endoscopic image. The manipulation assistance program causes the control device 5 to perform the step SF2-2 described above.

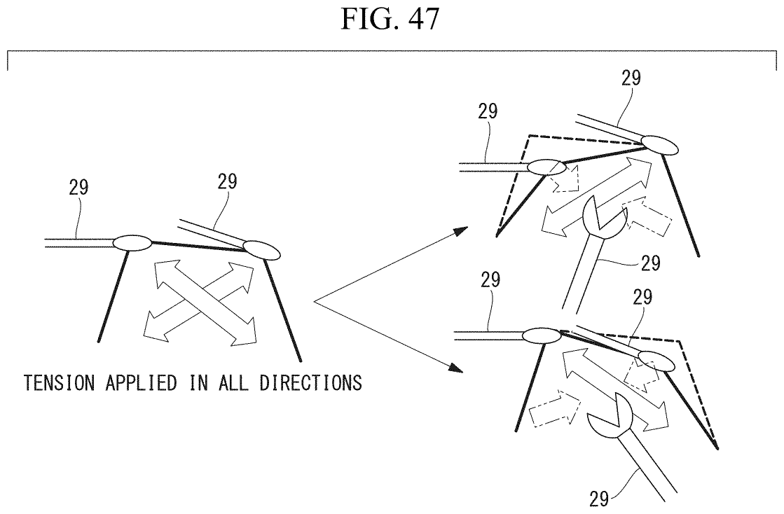

The open/close direction recognition step SF2-2 determines which of the forceps 29 of the assistant can relax the tissue tension in approximately the same direction as the direction in which the surgeon opens/closes the forceps 29. The open/close direction recognition step SF2-2 is performed by the evaluation unit 15. Since the surgeon can sufficiently grasp the living tissue if the living tissue is relaxed only in the direction in which the forceps 29 open and close, as shown in FIG. 47, which of the forceps 29 of the assistant to be moved during tissue relaxation is determined by the direction in which the surgeon's forceps 29 open/close.

The tissue relaxation navigation step SF3 and the tissue traction navigation step SF5 create a navigation to operate only one of the forceps 29 of the assistant determined by the open/close direction recognition step SF2-2. The navigation may, for example, be to present traction assistance information, such as an arrow indicating one of the forceps 29 to be operated, in the current endoscopic image.

According to this modification, the relaxation of the living tissue due to relaxation is minimized and the surgeon's grasp can be achieved more securely.

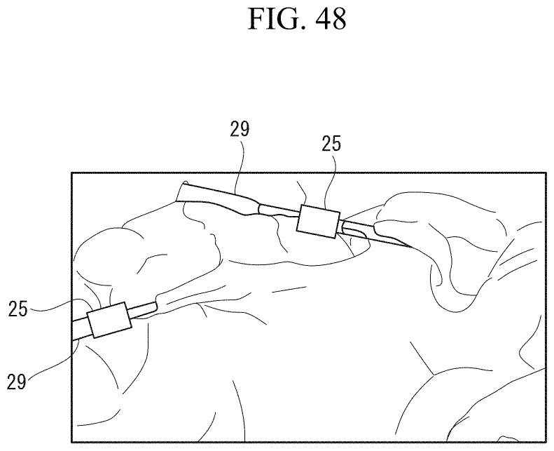

The fourth modification may, instead of recognizing the tension state from the endoscopic image, as shown in FIG. 48, for example, judge the tension state of the living tissue based on the sensor 25 mounted on the forceps 29 of the assistant. In this case, in the tension state determination step SF2 and the tissue traction navigation step SF5, after the amount of force during traction is measured by the sensor 25 mounted on the assistant's forceps 29, the evaluation unit 15 may navigate the relaxation of the living tissue and re-traction after the surgeon grasping operation, based on the measured values.

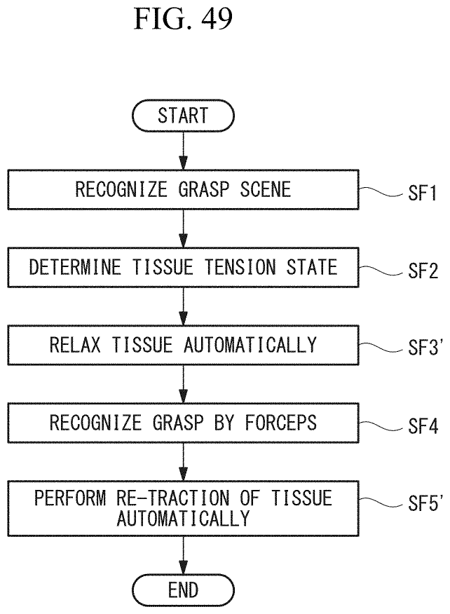

The fifth modification may include, in the case where the assistant uses forceps or manipulator having flexure joints driven with electric power or the like, instead of the tissue relaxation navigation step SF3 and the tissue traction navigation step SF5, an automatic tissue relaxation step SF3′ in which automatic relaxation operation is performed and an automatic tissue re-traction step SF5′ in which automatic re-traction operation is performed as shown in the flowchart of FIG. 49. The automatic tissue relaxation step SF3′ and the automatic tissue re-traction step SF5′ are performed by the evaluation unit 15 according to the manipulation assistance program.

According to this modification, semi-automation of the assistant's forceps or manipulator can be achieved.

### Fifth Embodiment

The endoscope system, manipulation assistance method, and manipulation assistance program according to the fifth embodiment of the present invention will be described below with reference to the accompanying drawings.

The endoscope system 1 according to this embodiment differs from the first to fourth embodiments in that it outputs information that supports the assistant to grasp, as assistance information.

In the description of this embodiment, the parts whose configurations are the same as in the endoscope system 1 of the first to fourth embodiments described above are denoted by the same reference numerals thereas, and their descriptions will be omitted. The image acquisition step, derivation step, and display step of the manipulation assistance program are the same as those of the first or second embodiment. The manipulation assistance program causes the control device 5 to execute the steps of the manipulation assistance method described below.

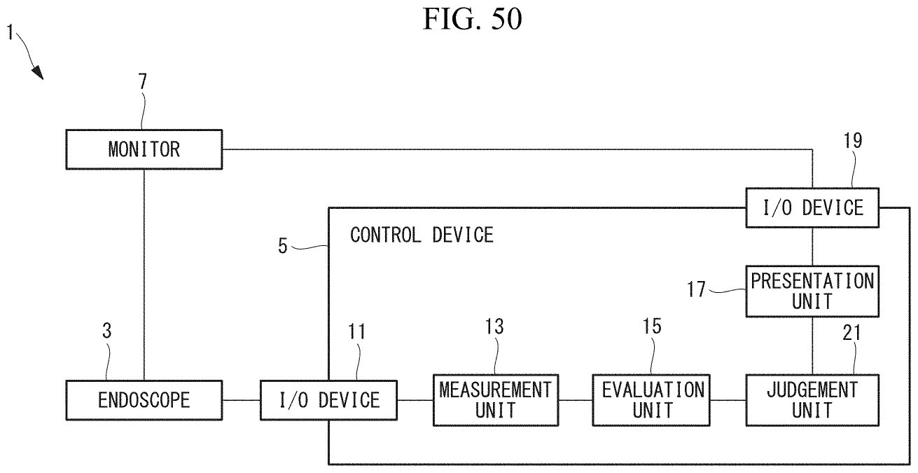

As shown in FIG. 50, the endoscope system 1 has a control device 5 including a measurement unit 13, an evaluation unit 15, a judgement unit 21, and a presentation unit 17.

The measurement unit 13 measures feature values related to the surgical scene and manipulation steps in the captured current endoscopic image.

The evaluation unit 15 evaluates the feature values measured by the measurement unit 13 using the model. To be specific, the evaluation unit 15 inputs the current endoscopic image to the model and thus recognizes the current surgical scene and manipulation steps based on the measurement results given by the measurement unit 13, and also evaluates, for example, whether or not the surgeon assists the assistant and type of assistance, based on the previous endoscopic images corresponding to the current surgical scene and manipulation steps.

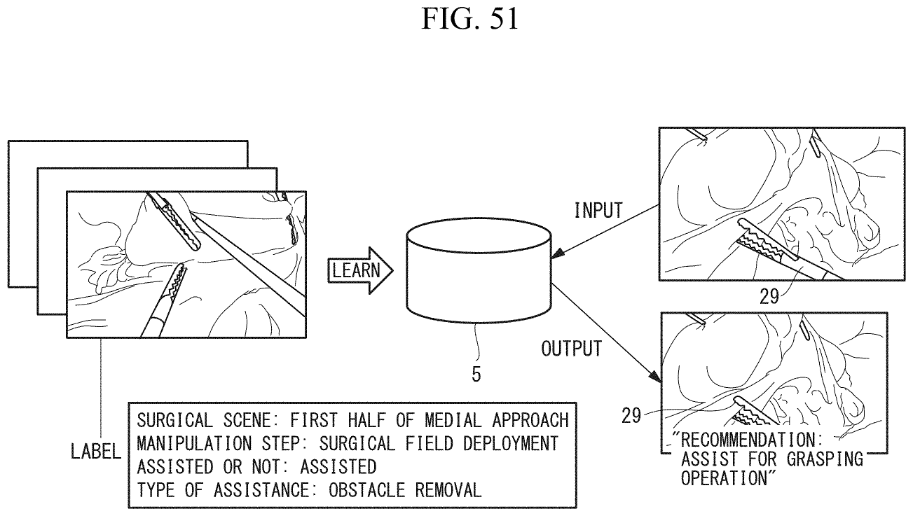

As shown in FIG. 51, for example, the model is trained with a plurality of previous endoscopic images labeled with the name of the surgical scene, the name of the manipulation step, whether or not the surgeon assists the assistant, and the type of assistance. In the example shown in FIG. 51, the previous endoscopic images learned by the model are labeled with Surgical scene: first half of medial approach, Manipulation step: surgical field deployment, Assisted or not: assisted, and Type of assistance: obstacle removal.

The judgement unit 21 judges the current surgical scene, manipulation step, whether it is assisted or not, and type of assistance based on the evaluation results given by the evaluation unit 15.

When the judgement unit 21 judges that assistance is to be performed, the presentation unit 17 constructs information that prompts the surgeon to assist (grasp assistance information) based on the type of assistance determined by the judgement unit 21, and then adds the constructed information that prompts assistance to the current endoscopic image. The information that prompts assistance indicates, for example, aside from the type of assistance, the work to be performed by the surgeon, such as tissue grasping and traction, and removal of obstacles such as the large intestine.

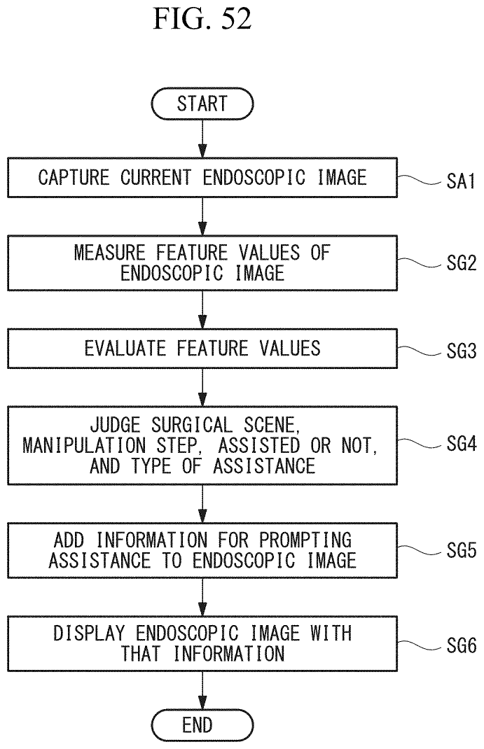

Next, the action of the endoscope system 1, manipulation assistance method, and manipulation assistance program in the aforementioned configuration will be explained with reference to the flowchart of FIG. 52.

To assist surgeon's manipulation using the endoscope system 1, manipulation assistance method, and manipulation assistance program according to this embodiment, when the endoscopic image of the living tissue acquired from the endoscope 3 is captured by the control device 5 (Step SA1), the measurement unit 13 measures the feature values of the endoscopic image captured by the control device 5 (Step SG2).

Next, the current endoscopic image is input to the model by the evaluation unit 15, and the current surgical scene and manipulation step are then recognized based on the measurement results given by the measurement unit 13. Subsequently, the evaluation unit 15 evaluates whether or not the surgeon assists the assistant, the type of assistance, and the like from the previous endoscopic images corresponding to the current surgical scene and manipulation step (Step SG3).

Next, the judgement unit 21 judges the current surgical scene, manipulation step, whether it is assisted or not, and the type of assistance based on the evaluation results given by the evaluation unit 15 (Step SG4). If it is judged that assistance is to be performed for grasping operation, the presentation unit 17 constructs, for example, the text “Recommendation: Assist for grasping operation” as information to prompt the surgeon to assist. The information that was constructed by the presentation unit 17 to prompt assistance is added to the current endoscopic image (Step SG5), and then sent to the monitor 7. As a result, “Recommendation: Assist for grasping operation” is presented in text in the current endoscopic image on the monitor 7 (Step SG6).

In endoscopic surgery, the surgeon and assistant work together to deploy the surgical field in order to perform the operation safely and efficiently. In such cases, there are frequent situations in which the assistant alone is unable to move the assistant's forceps to a position for grasping the tissue in order to perform the ideal surgical field deployment. In this case, for example, the surgeon needs to move the obstructing living tissue out of the way, or the assistant needs to move it to a position suitable for the surgical field deployment and where it is easy for the assistant to hold the living tissue.

Although these types of work would be easy for surgeons who are expert surgeons, if the surgeon is a resident or mid-career, the problem arises that he/she would not know where the assistant can easily grasp the tissue, or may not be able to move the living tissue to a position where the assistant can easily grasp the living tissue. Besides, if the surgeon mistakenly assumes that the assistant alone can grasp the living tissue, the problem arises that even when tissue movement, such as the removal of obstructive living tissue and traction of tissue necessary for the deployment of the surgical field, needs to be performed by the surgeon, it is not performed.

According to the endoscopy system 1, manipulation assistance method, and manipulation assistance program, the need for assistance by the surgeon can be easily recognized in real time by extracting information from a model that has learned previous surgical data. Aside from that, based on the extracted information, the necessary assistance information is presented in association with the current endoscopic image, thereby enabling smooth operation without stopping the surgical flow independently of the skill of the surgeon.

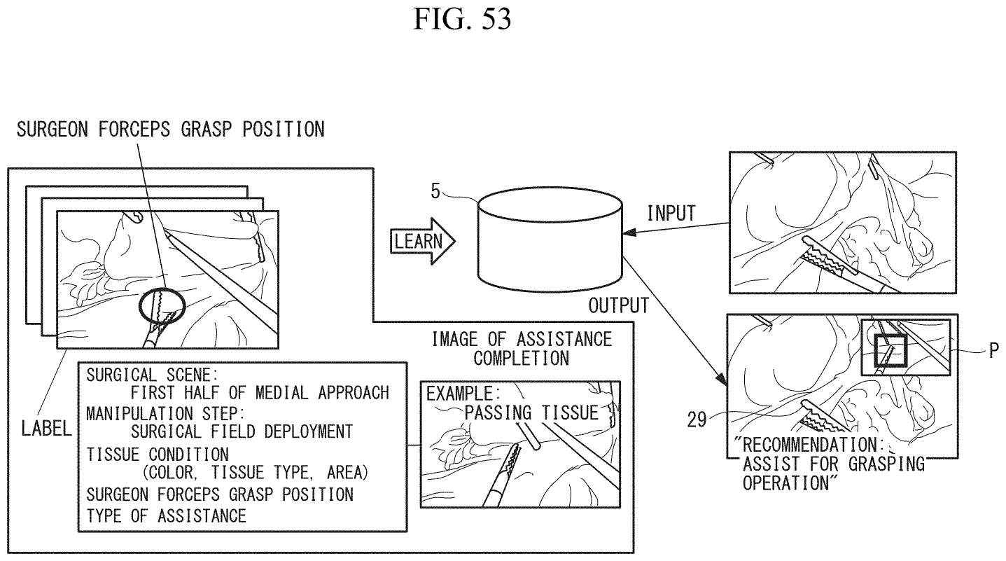

This embodiment can be modified in the following manner. In the first modification, for example, as shown in FIG. 53, an image P of a scene in which assistance is provided to an assistant in a similar case may be output as assistance information.

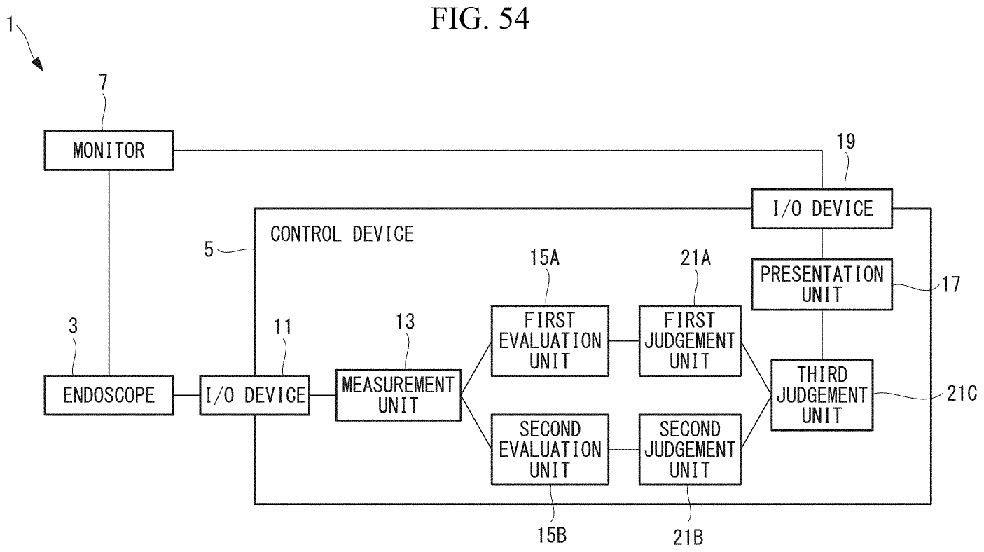

In this modification, as shown in FIG. 54, the control device 5 includes, instead of the evaluation unit 15 and the judgement unit 21, a first evaluation unit 15A and a first judgement unit 21A, a second evaluation unit 15B and a second judgement unit 21B, and a third judgement unit 21C.

As shown in FIG. 53, the first and second evaluation units 15A and 15B uses a model trained with a plurality of previous endoscopic images labeled with the name of the surgical scene, the name of the manipulation step, the tissue condition, such as the type, color, and area of the visible living tissue, the positions that the surgeon grasps with the forceps 29, and the type of assistance, and associated with the image obtained at the completion of assistance for each assistant.

The first evaluation unit 15A inputs the current endoscopic image to the model and thus evaluates the surgical scene and manipulation step based on the feature values measured by the measurement unit 13.

The first judgement unit 21A judges the current surgical scene and manipulation step based on the results of the evaluation by the first evaluation unit 15A.

The second evaluation unit 15B inputs the current endoscopic image to the model and thus evaluates the tissue condition and the positions that the surgeon grasps with the forceps 29 based on the feature values measured by the measurement unit 13.

The second judgement unit 21B judges the current tissue condition and the positions that the surgeon grasps with the forceps 29 based on the results of the evaluation by the second evaluation unit 15B.

The third judgement unit 21C judges the type of assistance needed for the assistant based on the results of judgment by the first judgement unit 21A and the results of judgment by the second judgement unit 21B, and extracts the image P of the scene where the assistance for the assistant is performed in a similar case.

Based on the results of judgment by the third judgement unit 21C, the presentation unit 17 constructs grasp assistance information, such as text indicating the recommended type of assistance, and then adds the constructed text and the image P from a similar case extracted by the third judgement unit 21C, to the current endoscopic image. As a result, on the monitor 7, as shown in FIG. 53, the text “Recommendation: Assist for grasping operation” indicating the type of recommended assistance and the image P from a similar case are presented in the current endoscopic image.

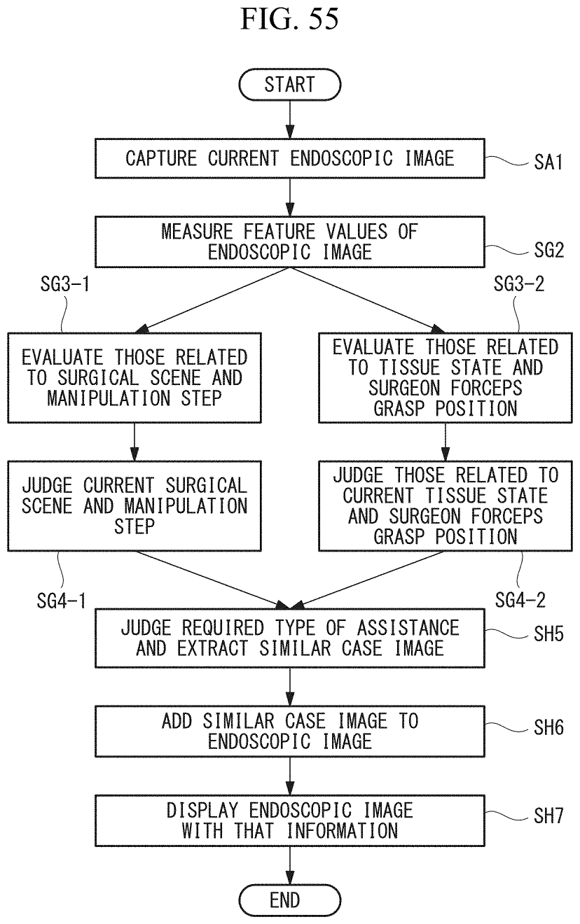

In this modification, as shown in the flowchart of FIG. 55, the current endoscopic image obtained when the surgeon grasps the living tissue for field deployment is input to the model, so that the current surgical scene, manipulation step, tissue condition, and positions that the surgeon grasps with the forceps 29 are evaluated and judged (Steps SG3-1, SG3-2, SG4-1, and SG4-2). The type of assistance that will be required is then judged and the image P from a similar case is extracted (Step SH5), so that text indicating the recommended type of assistance and the image P from the similar case are presented in the current endoscopic image (Steps SH6 and SH7). If the image P of from the similar case differs from the surgeon's image, inputting this fact through an input device may further present images P from, for example, the second and third candidate similar cases.

According to this modification, presenting the image P from a similar case together with the current endoscopic image allows the surgeon to recognize, from the current grasping position, to which position the living tissue should be moved and pulled with what kind of assistance to the assistant. There is also the side benefit of allowing the assistant to also recognize in which position the living tissue should be received.

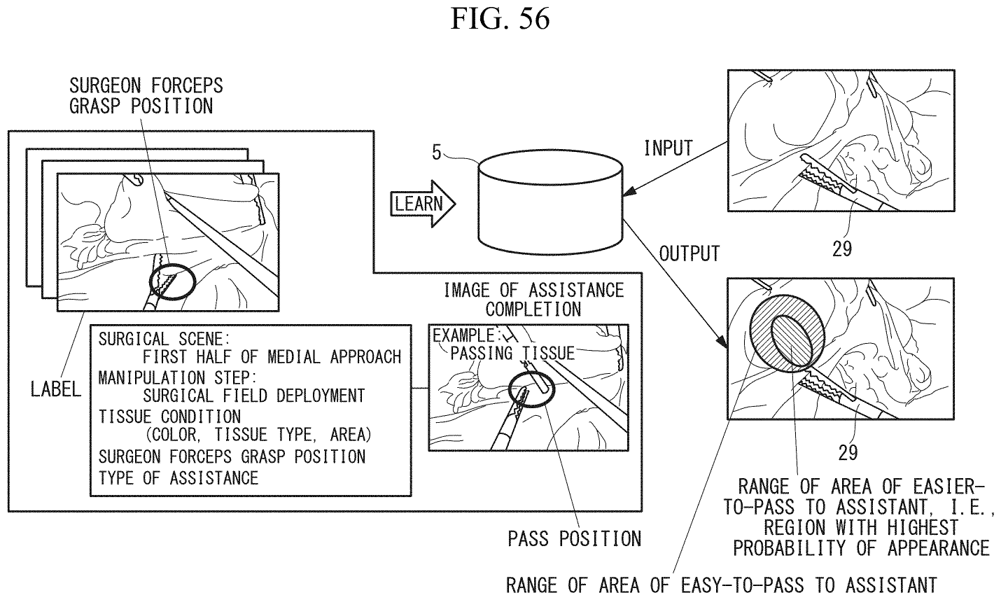

In the second modification, for example, as shown in FIG. 56, information indicating a range of area of easy-to-pass to an assistant used for assistant grasp support may be presented as grasp assistance information.

The first evaluation unit 15A and the second evaluation unit 15B use a model trained with a plurality of previous endoscopic images labeled with the name of the surgical scene, the name of the manipulation step, the tissue condition, such as the type, color, and area of the visible living tissue, the positions that the surgeon grasps with the forceps 29, and the type of assistance, and trained with the positions of the forceps 29 of the assistant obtained when the surgeon passes the living tissue to the assistant, which is a post-scene in each previous endoscopic image.

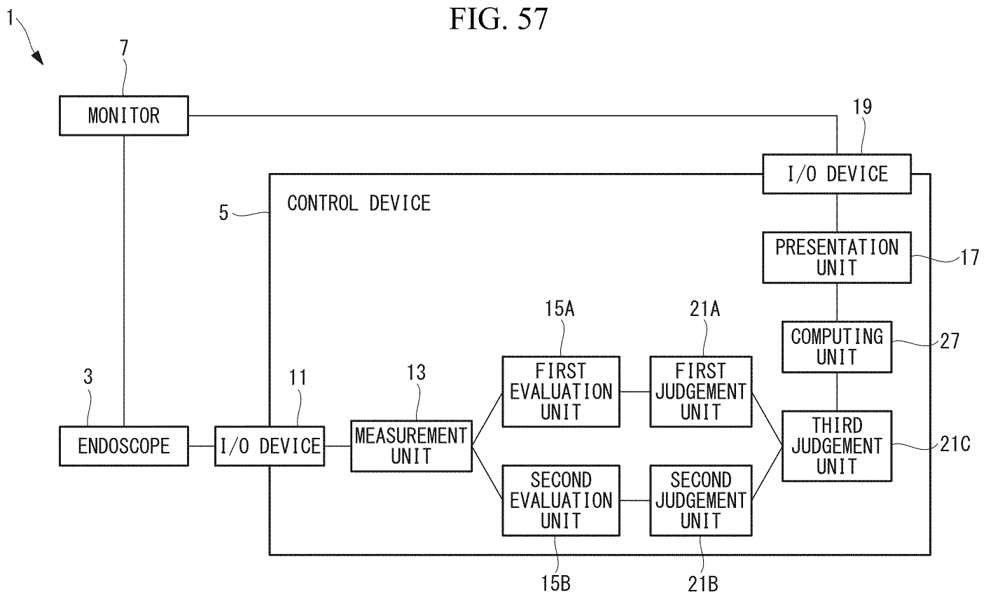

In this modification, as shown in FIG. 57, the control device 5 further includes a computing unit 27 besides the configuration of the first modification. The computing unit 27 calculates the probability of the presence of the position of the forceps 29 of the surgeon in an image P from a similar case obtained at the time of assistance to the assistant extracted by the third judgement unit 21C.

The presentation unit 17 constructs a probability distribution of the position of the forceps 29 of the surgeon at the time of completion of assisting the assistant, as grasp assistance information based on the results of computation by the computing unit 27. As shown in FIG. 56, regarding the probability distribution, grasp assistance information may be presented in the current endoscopic image by, for example, painting in the region where the surgeon's forceps 29 exist at the completion of assisting the assistant in a similar case, and each region may be color-coded based on a predetermined threshold of the probability of the appearance of the surgeon's forceps 29. In this way, a range of area of most easy-to-pass to the assistant, i.e., the region with the highest probability of the appearance of the surgeon's forceps 29, and the area of second most easy-to-pass to the assistant are color-coded and presented.

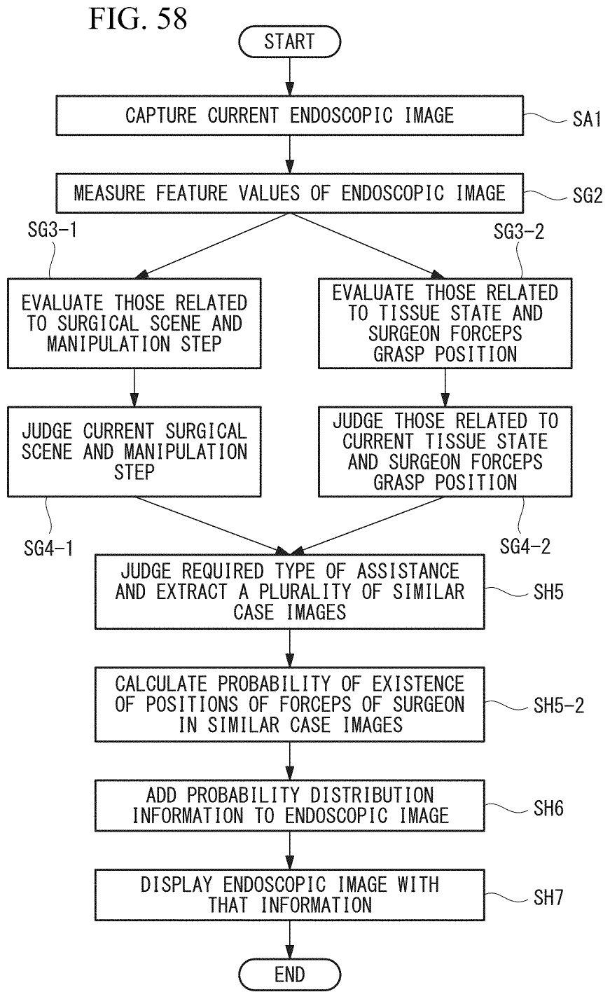

In this modification, as shown in the flowchart of FIG. 58, the current endoscopic image obtained when the surgeon grasps the living tissue for field deployment is input to the model, so that the probability of existence of the position of the forceps 29 of the surgeon in the image P from a similar case is calculated from the current surgical scene, manipulation step, tissue condition, and the positions that the surgeon grasps with the forceps 29 (Step SH5-2). The probability distribution of the position of the surgeon's forceps 29 at the completion of assisting the assistant is then presented in the current endoscopic image as grasp assistance information (Steps SH6 and SH7).

According to this modification, showing the distribution of forceps positions observed at the time of completion of assistance in a similar previous case as assistance information allows the surgeon to easily recognize to which position from the current grasping position the living tissue should be moved in order to provide effective assistance to the assistant.

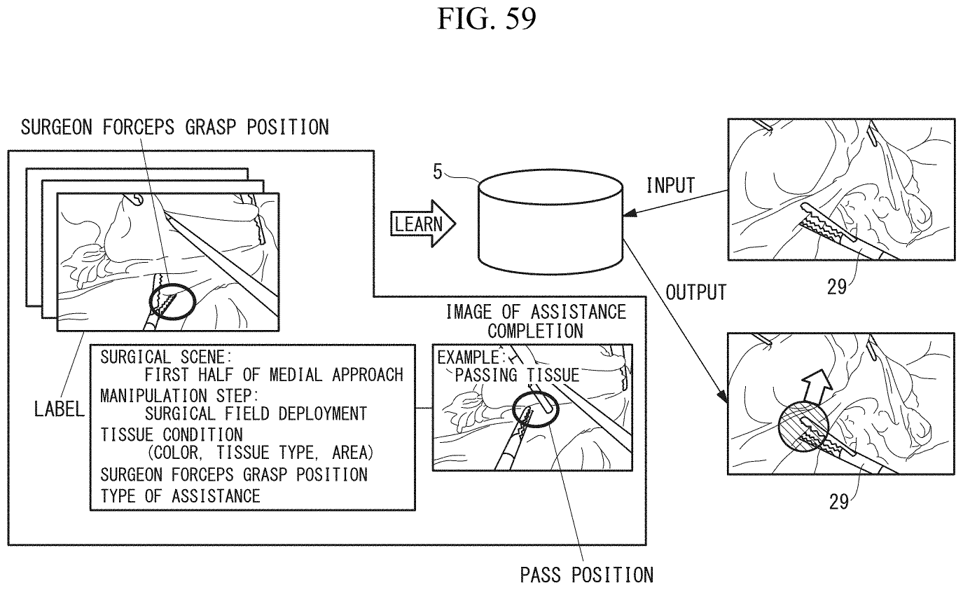

In the third modification, as shown in FIG. 59, for example, the grasping position and traction direction during the tissue manipulation by the surgeon for assistant grasping support may be presented as assistance information (traction assistance information and grasp assistance information). The configuration of this modification is the same as in the second modification.

The first evaluation unit 15A and the second evaluation unit 15B use a model trained with a plurality of previous endoscopic images labeled with the name of the surgical scene, the name of the manipulation step, the tissue condition, such as the type, color, and area of the visible living tissue, the positions that the surgeon grasps with the forceps 29, and the type of assistance, and trained with the position of the forceps 29 of the surgeon obtained at the time of completion of assistance to the assistant, which is a post-scene in each previous endoscopic image.

Based on the probability of existence of the position of the surgeon's forceps 29 in the image P from a similar case at the time of assisting the assistant calculated by the computing unit 27, the presentation unit 17 recognizes the difference between the position with the highest existence probability of the surgeon's forceps 29 from the similar case and the current position of the surgeon's forceps 29. Then, the presentation unit 17 constructs arrows or the like as assistance information indicating the direction of operation of the surgeon's forceps 29 in which the recognized difference is eliminated.

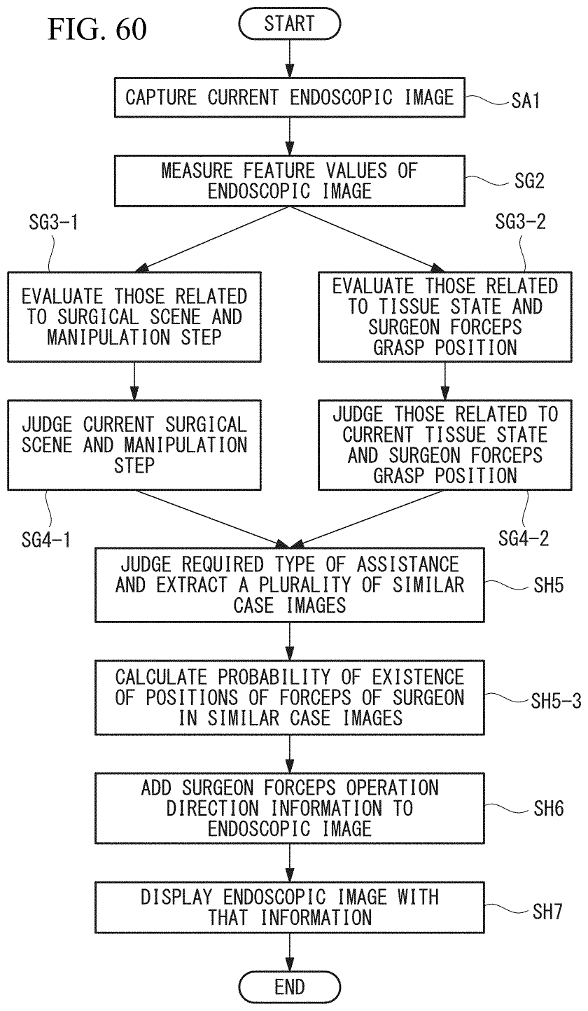

In this modification, as shown in the flowchart of FIG. 60, the current endoscopic image obtained when the surgeon grasps the living tissue for field deployment is input to the model, so that the probability of existence of the position of the forceps 29 of the surgeon in the image P from a similar case is calculated from the current surgical scene, manipulation step, tissue condition, and the positions that the surgeon grasps with the forceps 29 (Step SH5-3). The direction in which the surgeon's forceps 29 are moved for assisting the assistance is then presented in the current endoscopic image (Steps SH6 and SH7).

According to this modification, the surgeon recognizes where to move the living tissue from the current grasping position to assist the assistant without a failure. This improves the efficiency and safety of the surgery.

Each of the aforementioned embodiments, which illustrates and explains the case of displaying the derived grasp assistance information and traction assistance information through the monitor 7, may use grasp assistance information and traction assistance information for other applications instead. For example, based on the grasp assistance information or traction assistance information, the energy output of an energy device (not shown in the drawings) may be adjusted, or the traction strength and traction direction for the manipulator of a robot (not shown in the drawings) may be adjusted.

As an example, the adjustment of the energy output of an energy device will be explained below.

An energy device is, for example, a device that outputs energy in the form of, for example, high-frequency power or ultrasound to perform treatment such as coagulation, sealing, hemostasis, incision, dissection, or separation of the living tissue adjoining the output unit for outputting that energy. The energy device is, for example, a monopolar device in which high-frequency power passes between an electrode at the tip of the device and an electrode outside the body; a bipolar device in which high-frequency power passes between two jaws; an ultrasonic device equipped with a probe and jaw in which the probe vibrates ultrasonically; and a combination device in which high-frequency power passes between the probe and jaws and the probe vibrates ultrasonically.

In general, one of the keys to performing energy treatment in surgery is to avoid thermal damage to surrounding organs by controlling thermal diffusion from the energy device. However, as the tissues to be treated are not uniform, there are variations in the time required for treatment such as dissection due to variations in tissue type, variations in tissue condition, and variations among individual patients, and the degree of thermal diffusion also varies. To cope with these variations in the time required for the treatment and the degree of thermal diffusion and suppress thermal diffusion, surgeons adjust the amount of tissue grasping and tissue tension, which is however an operation that requires experience and may be difficult especially for non-experts to achieve suitable adjustment.

Thus, in the case of treatment using an energy device, as thermal diffusion to the surroundings is often a problem, the surgeon performs the treatment while estimating the degree of diffusion. For example, there are known techniques to recognize the tissue type, such as vascular or non-vascular, based on the energy output data on the energy device. However, the degree of thermal diffusion is not only classified into two categories: vascular and non-vascular, but is also affected by the tissue condition, such as tissue thickness and immersion in blood, or the amount of grasping by the device or operation by the surgeon such as traction strength. To be specific, thermal diffusion occurs when the heat in the tissue caused by energy application by the energy device diffuses into or onto the surrounding tissue. Alternatively, thermal diffusion occurs when the energy output by the energy device diffuses into the surrounding tissue, and the surrounding tissue into which the energy has diffused generates heat. The degree of thermal diffusion depends on the tissue type, tissue condition, amount of tissue grasp, or tissue tension.

In this modification, the processor 14 executes the treatment according to the program so that the endoscope system 1 may apply the suitable energy to the tissue according to the grasp assistance information or traction assistance information. This can reduce the thermal diffusion from the target of treatment by the energy device to the surrounding tissue. Besides, the burden on the surgeon can be reduced with the endoscope system 1 that performs adjustment of the energy output instead of adjustment of the grasping amount and tension which would conventionally be performed by the surgeon. In addition, as the endoscope system 1 adjusts the output autonomously, even inexperienced surgeons can perform stable treatment. Consequently, the stability of the operation can be improved or manipulation equalization can be achieved independently of the experience of the surgeon.

An example of treatment for recognizing tissue tension will be explained below.

When an endoscopic image is input to the control device 5, the control device 5 recognizes tissue tension from the endoscopic image by executing a tissue recognition program adjusted by machine learning. Tissue tension is the tension applied to the tissue grasped by the jaws of the bipolar device. This tension is generated by the traction of the tissue by the bipolar device or by the traction of the tissue by an instrument such as forceps. Appropriate tension applied to the tissue allows for proper treatment with the energy device. However, if the tension is unsuitable tissue tension, for example, weak, the treatment with the energy device takes longer and is more likely to cause thermal diffusion. The control device 5 recognizes the score, which is an evaluation value of the tension applied to the tissue grasped by the jaw, from the endoscopic image. The control device 5 then compares the score of the tissue tension recognized from the endoscopic image with the threshold and outputs the result of the comparison.

Next, the control device 5 instructs to change the output according to the tissue tension recognized from the endoscopic image. For example, the main memory 16 stores table data associated with an energy output adjustment instruction for each tissue type. Referring to that table data, the control device 5 adjusts the output sequence of the bipolar device by outputting an energy output adjustment instruction corresponding to the tissue type. The algorithm for outputting the energy output adjustment instruction according to the tissue type is not necessarily the aforementioned one.

The teacher data in the learning phase includes images of various tissue tensions as training images. The teacher data also includes, as correct data, the evaluation regions for which scores are to be calculated and the scores calculated from the images of the evaluation regions.

An example of the first recognition treatment performed when the control device 5 recognizes tissue tension will be explained below. The control device 5 outputs a tissue tension score by estimating the tissue tension by regression.

To be specific, the control device 5 detects, from the endoscopic image, evaluation regions that are subject to tension evaluation by image recognition processing with a learned model, and estimates the tissue tension from the images in these evaluation regions. The control device 5 outputs a high score when the suitable tension is applied to the tissue in the treatment captured in the endoscopic image. In the learning phase, the learning device (not shown in the drawings) generates a learned model using the endoscopic image accompanied with the information specifying the evaluation region and the tissue tension score as the teacher data. Alternatively, the teacher data may be a video, i.e., a time-series image. For example, the video shows a tissue traction operation with an energy device or forceps and is associated with an evaluation region and a score. The score is quantified, for example, based on hue, saturation, brightness, luminance, or information on tissue movement due to traction of the tissue in the endoscopic image or video. The score obtained from the quantification is assigned to each endoscopic image or video for training.

The following will explain an example of the second recognition processing performed when the control device 5 recognizes tissue tension. The control device 5 detects the tip of the instrument by detection, sets an evaluation region based on the detection result, and estimates the tissue tension by regression for the image within the evaluation region.

To be specific, the control device 5 includes a first learned model for detecting the instrument tip and a second learned model for estimating tissue tension. The control device 5 detects the position of the jaws from the endoscopic image by image recognition processing using the first learned model. The control device 5 sets an evaluation region around the jaws according to a predetermined rule based on the detected position of the jaws. The predetermined rule is, for example, setting the area within a predetermined distance from the position of the jaws as the evaluation region. In the learning phase, the learning device generates the first learned model, using the endoscopic image annotated with the position of the device tip, i.e., the position of the jaws of the bipolar device, as the teacher data. The control device 5 outputs a tissue tension score, estimating the tissue tension from the image within the evaluation region by image recognition processing using the second learned model. In the learning phase, the learning device generates a learned model using the endoscopic image or video accompanied with the tissue tension score as the teacher data.

Although the embodiments of the present invention have been described in detail with reference to the accompanying drawings, the specific configuration is not limited by these embodiments, and involves design changes and the like without departing from the scope of the present invention. For example, the present invention is not limited to the aforementioned embodiments and modifications, but may be applied to embodiments in which these embodiments and modifications are used in an appropriate combination. For example, the grasp assistance information based on one of the embodiments with the traction assistance information based on another embodiment may be combined and both may be associated with the endoscopic image and presented. In addition, although the aforementioned embodiments illustrate and explain the case in which the grasp assistance information and traction assistance information are displayed on the monitor 7, displaying the information on the monitor 7 may be accompanied by a sound notice.

The following aspects can be also derived from the embodiments.

A first aspect of the present invention is an endoscope system including: an endoscope that images living tissue to be treated by at least one instrument; and a control device that includes a processor configured to derive at least one of the following information: grasp assistance information on a grasping operation to grasp the living tissue by the at least one instrument, and traction assistance information on a traction operation for traction of the living tissue by the at least one instrument, based on an endoscopic image acquired by the endoscope.

According to this aspect, when the endoscope acquires an endoscopic image of the living tissue to be treated by the at least one instrument, at least one of the following information: the grasp assistance information and traction assistance information related to the living tissue given by the at least one instrument is derived by the processor of the control device. This allows the surgeon to perform grasping and traction operations according to at least one of the following information: the grasp assistance information and the traction assistance information, thereby improving the stability of the manipulation operation and equalizing the manipulation independently of the surgeon's experience.

The endoscope system according to this aspect may further includes a display device that displays at least one of the following information: the grasp assistance information and the traction assistance information derived by the control device, in association with the endoscopic image.

This makes it possible to easily recognize the grasp assistance information or traction assistance information while viewing the living tissue in the endoscopic image displayed by the display device.

In the endoscope system according to this aspect, the processor may be configured to derive both the grasp assistance information and the traction assistance information, and the display device may display both the grasp assistance information and the traction assistance information, in association with the endoscopic image.

This configuration allows the surgeon to perform grasping and traction operations according to both the grasp assistance information and the traction assistance information, thereby improving the stability of the manipulation operation and equalizing the manipulation independently of the surgeon's experience.

In the endoscope system according to this aspect, the processor may be configured to set an evaluation region in the endoscopic image of the traction operation being performed, evaluate the traction operation in the evaluation region, and output an evaluation result as the traction assistance information.

With this configuration, the evaluation results by the processor for the traction operation contribute to unification of the evaluation criteria for the traction operation, which would conventionally depend on the experience of each surgeon. Consequently, the surgeon pulls the living tissue according to the traction assistance information, which reduces variations in manipulation operations by the surgeon and further equalizes the manipulation operation.

In the endoscope system according to this aspect, the processor may be configured to evaluate the traction operation from changes in feature values of the living tissue in the evaluation region

When living tissue is pulled, the feature values of the living tissue extracted from the endoscopic image change. Thus, the traction operation can be evaluated by image processing by the processor.

The endoscope system according to this aspect may output the evaluation result as a score.

When the evaluation results are displayed as scores, the level of difference from a suitable traction operation can be easily recognized.

In the endoscope system according to this aspect, the processor may be configured to evaluate the traction operation from a change in a linear component of capillaries of the living tissue in the evaluation region.

When living tissue is pulled, the linear component of capillaries in the living tissue increases. For this reason, the traction operation can be accurately evaluated based on the amount of change in the linear component of capillaries before and after traction.

In the endoscope system according to this aspect, the at least one instrument may include a plurality of instruments, and the processor may be configured to evaluate the traction operation from a rate of change before and after traction of a distance between portions of the living tissue grasped by the plurality of instruments in the evaluation region.

With the living tissue grasped by the plurality of instruments, pulling the living tissue in different directions depending on each instrument increases the distance between the grasped portions of the living tissue. If the distance between the grasped portions after traction relative to the distance between the grasped portions before traction is within a predetermined range of increase, the traction operation can be determined as being suitable. Therefore, the traction operation can be accurately evaluated based on the rate of change of the distance between the grasped portions before and after traction.

In the endoscope system according to this aspect, the at least one instrument may include a plurality of instruments, and the processor may be configured to evaluate the traction operation from, as the changes in the feature values of the living tissue, at least one of the following information: a change in a linear component of capillaries of the living tissue in the evaluation region, a rate of change before and after traction of a distance between portions of the living tissue grasped by the plurality of instruments in the evaluation region, an amount of movement of the living tissue in the evaluation region, a change in a vascular density of the living tissue in the evaluation region, a change in an area of a bright spot in the evaluation region, a change in a spatial frequency of the living tissue in the evaluation region, a change in a shape of a color histogram in the evaluation region, a change in a shape of an edge of an incision region of the living tissue in the evaluation region, a change in an area or contour shape of a region enclosed by each position of the living tissue grasped by the plurality of instruments in the evaluation region, and a change in a texture of a surface of the living tissue in the evaluation region.

In the endoscope system according to this aspect, when the evaluation result is less than a preset threshold, the processor may be configured to output, as the traction assistance information, a traction direction in which the evaluation result becomes greater than the threshold.

This configuration allows the surgeon to achieve a suitable traction operation in which the evaluation result is greater than the threshold, by further pulling the living tissue in the traction direction displayed as the traction assistance information.

In the endoscope system according to this aspect, the processor may be configured to set, as the evaluation region, a region including a fixed line where a position of the living tissue recognized from the endoscopic image remains unchanged and a position of the living tissue grasped by the at least one instrument.

This configuration allows the evaluation region to be set based on the actual grasped position.

In the endoscope system according to this aspect, the processor may be configured to evaluate the traction operation from an angle between a longitudinal axis of the at least one instrument and the fixed line in the endoscopic image.

This configuration allows for evaluation of the traction operation by computing processing by the processor, which speeds up the processing.

In the endoscope system according to this aspect, the processor may be configured to recognize a surgical scene based on the endoscopic image and output, as the grasp assistance information, a grasp target tissue in the surgical scene.

With this configuration, the surgeon performing a grasp operation according to the grasp assistance information can correctly grasp the necessary living tissue according to the surgical scene and properly perform the subsequent traction operation. This also eliminates the need for grasping living tissue that does not need to be grasped and reduces the risk of damaging the living tissue.

In the endoscope system according to this aspect, the processor may be configured to derive a grasp amount of the living tissue being grasped by the at least one instrument based on the endoscopic image, and output, as the grasp assistance information, the derived grasp amount.

This configuration allows the surgeon to recognize whether or not the living tissue is sufficiently grasped, and enables the grasping operation with a suitable amount of grasp.

A second aspect of the present invention is a manipulation assistance method including deriving, based on a living tissue image of living tissue to be treated by an at least one instrument, grasp assistance information on a grasping operation to grasp the living tissue by the at least one instrument, and traction assistance information on a traction operation for traction of the living tissue by the at least one instrument.

The manipulation assistance method according to this aspect, including: setting an evaluation region in the living tissue image of the traction operation being performed; evaluating the traction operation in the set evaluation region; and outputting an evaluation result as the traction assistance information.

In the manipulation assistance method according to this aspect, including evaluating the traction operation from changes in feature values of the living tissue in the evaluation region.

In the manipulation assistance method according to this aspect, the at least one instrument may include a plurality of instruments, and the method includes evaluating the traction operation from, as the changes in the feature values of the living tissue, at least one of the following information: a change in a linear component of capillaries of the living tissue in the evaluation region, a rate of change before and after traction of a distance between portions of the living tissue grasped by the plurality of instruments in the evaluation region, an amount of movement of the living tissue in the evaluation region, a change in a vascular density of the living tissue in the evaluation region, a change in an area of a bright spot in the evaluation region, a change in a spatial frequency of the living tissue in the evaluation region, a change in a shape of a color histogram in the evaluation region, a change in a shape of an edge of an incision region of the living tissue in the evaluation region, a change in an area or contour shape of a region enclosed by each position of the living tissue grasped by the plurality of instruments in the evaluation region, and a change in a texture of a surface of the living tissue in the evaluation region.

A third aspect of the present invention is a manipulation assistance program that causes a computer to execute: an acquisition step of acquiring an image of living tissue to be treated by at least one instrument; and a derivation step of deriving, based on the acquired living tissue image, at least one of the following information: grasp assistance information on a grasping operation to grasp the living tissue by the at least one instrument, and traction assistance information on a traction operation for traction of the living tissue by the at least one instrument.

In the manipulation assistance program according to this aspect, the derivation step may set an evaluation region in the living tissue image of the traction operation being performed; evaluate the traction operation in the set evaluation region; and output an evaluation result as the traction assistance information.

In the manipulation assistance program according to this aspect, the derivation step may evaluate the traction operation from changes in feature values of the living tissue in the evaluation region.

In the manipulation assistance program according to this aspect, the at least one instrument may include a plurality of instruments, and the derivation step may evaluate the traction operation from, as the changes in the feature values of the living tissue, at least one of the following information: a change in a linear component of capillaries of the living tissue in the evaluation region, a rate of change before and after traction of a distance between portions of the living tissue grasped by the plurality of instruments in the evaluation region, an amount of movement of the living tissue in the evaluation region, a change in a vascular density of the living tissue in the evaluation region, a change in an area of a bright spot in the evaluation region, a change in a spatial frequency of the living tissue in the evaluation region, a change in a shape of a color histogram in the evaluation region, a change in a shape of an edge of an incision region of the living tissue in the evaluation region, a change in an area or contour shape of a region enclosed by each position of the living tissue grasped by the plurality of instruments in the evaluation region, and a change in a texture of a surface of the living tissue in the evaluation region.

### REFERENCE SIGNS LIST

1 Endoscope system
3 Endoscope
5 Control device
7 Monitor (display device)
9 Jaws (instrument)
14 Processor
29 Forceps (instrument)

1. An endoscope system comprising:
an endoscope that images living tissue to be treated by at least one instrument; and
a control device that includes a processor configured to derive at least one of the following information: grasp assistance information on a grasping operation to grasp the living tissue by the at least one instrument, and traction assistance information on a traction operation for traction of the living tissue by the at least one instrument, based on an endoscopic image acquired by the endoscope.

2. The endoscope system according to claim 1, further comprising a display device that displays at least one of the following information: the grasp assistance information and the traction assistance information derived by the control device, in association with the endoscopic image.

3. The endoscope system according to claim 1, wherein
the processor is configured to derive both the grasp assistance information and the traction assistance information, and
the display device displays both the grasp assistance information and the traction assistance information, in association with the endoscopic image.

4. The endoscope system according to claim 1, wherein the processor is configured to set an evaluation region in the endoscopic image of the traction operation being performed, evaluate the traction operation in the evaluation region, and output an evaluation result as the traction assistance information.

5. The endoscope system according to claim 4, wherein the processor is configured to evaluate the traction operation from changes in feature values of the living tissue in the evaluation region.

6. The endoscope system according to claim 5, wherein
the at least one instrument comprises a plurality of instruments, and
the processor is configured to evaluate the traction operation from, as the changes in the feature values of the living tissue, at least one of the following information: a change in a linear component of capillaries of the living tissue in the evaluation region, a rate of change before and after traction of a distance between portions of the living tissue grasped by the plurality of instruments in the evaluation region, an amount of movement of the living tissue in the evaluation region, a change in a vascular density of the living tissue in the evaluation region, a change in an area of a bright spot in the evaluation region, a change in a spatial frequency of the living tissue in the evaluation region, a change in a shape of a color histogram in the evaluation region, a change in a shape of an edge of an incision region of the living tissue in the evaluation region, a change in an area or contour shape of a region enclosed by each position of the living tissue grasped by the plurality of instruments in the evaluation region, and a change in a texture of a surface of the living tissue in the evaluation region.

7. The endoscope system according to claim 4, wherein the processor is configured to output the evaluation result as a score.

8. The endoscope system according to claim 4, wherein when the evaluation result is less than a preset threshold, the processor is configured to output, as the traction assistance information, a traction direction in which the evaluation result becomes greater than the threshold.

9. The endoscope system according to claim 4, wherein the processor is configured to set, as the evaluation region, a region including a fixed line where a position of the living tissue recognized from the endoscopic image remains unchanged and a position of the living tissue grasped by the at least one instrument.

10. The endoscope system according to claim 9, wherein the processor is configured to evaluate the traction operation from an angle between a longitudinal axis of the at least one instrument and the fixed line in the endoscopic image.

11. The endoscope system according to claim 1, wherein the processor is configured to recognize a surgical scene based on the endoscopic image and output, as the grasp assistance information, a grasp target tissue in the surgical scene.

12. The endoscope system according to claim 1, wherein the processor is configured to derive a grasp amount of the living tissue being grasped by the at least one instrument based on the endoscopic image, and output, as the grasp assistance information, the derived grasp amount.

13. A manipulation assistance method comprising deriving, based on a living tissue image of living tissue to be treated by an at least one instrument, at least one of the following information: grasp assistance information on a grasping operation to grasp the living tissue by the at least one instrument, and traction assistance information on a traction operation for traction of the living tissue by the at least one instrument.

14. The manipulation assistance method according to claim 13, comprising:
setting an evaluation region in the living tissue image of the traction operation being performed;
evaluating the traction operation in the set evaluation region; and
outputting an evaluation result as the traction assistance information.

15. The manipulation assistance method according to claim 14, comprising evaluating the traction operation from changes in feature values of the living tissue in the evaluation region.

16. The manipulation assistance method according to claim 15, wherein
the at least one instrument comprises a plurality of instruments, and
the method comprises evaluating the traction operation from, as the changes in the feature values of the living tissue, at least one of the following information: a change in a linear component of capillaries of the living tissue in the evaluation region, a rate of change before and after traction of a distance between portions of the living tissue grasped by the plurality of instruments in the evaluation region, an amount of movement of the living tissue in the evaluation region, a change in a vascular density of the living tissue in the evaluation region, a change in an area of a bright spot in the evaluation region, a change in a spatial frequency of the living tissue in the evaluation region, a change in a shape of a color histogram in the evaluation region, a change in a shape of an edge of an incision region of the living tissue in the evaluation region, a change in an area or contour shape of a region enclosed by each position of the living tissue grasped by the plurality of instruments in the evaluation region, and a change in a texture of a surface of the living tissue in the evaluation region.

17. A non-transitory computer-readable medium having a manipulation assistance program stored therein, the program causing a computer to execute functions of:
acquiring an image of living tissue to be treated by at least one instrument; and
deriving, based on the acquired living tissue image, at least one of the following information: grasp assistance information on a grasping operation to grasp the living tissue by the at least one instrument, and traction assistance information on a traction operation for traction of the living tissue by the at least one instrument.

18. The non-transitory computer-readable medium according to claim 17, wherein in the deriving, setting an evaluation region in the living tissue image of the traction operation being performed; evaluating the traction operation in the set evaluation region; and outputting an evaluation result as the traction assistance information.

19. The non-transitory computer-readable medium according to claim 18, wherein in the deriving, evaluating the traction operation from changes in feature values of the living tissue in the evaluation region.

20. The manipulation assistance program according to claim 19, wherein
the at least one instrument comprises a plurality of instruments, and
in the deriving, evaluating the traction operation from, as the changes in the feature values of the living tissue, at least one of the following information: a change in a linear component of capillaries of the living tissue in the evaluation region, a rate of change before and after traction of a distance between portions of the living tissue grasped by the plurality of instruments in the evaluation region, an amount of movement of the living tissue in the evaluation region, a change in a vascular density of the living tissue in the evaluation region, a change in an area of a bright spot in the evaluation region, a change in a spatial frequency of the living tissue in the evaluation region, a change in a shape of a color histogram in the evaluation region, a change in a shape of an edge of an incision region of the living tissue in the evaluation region, a change in an area or contour shape of a region enclosed by each position of the living tissue grasped by the plurality of instruments in the evaluation region, and a change in a texture of a surface of the living tissue in the evaluation region.
